(19) United States
(12) Patent Application Publication
(10) Pub. No.: US 2024/0139420 A1
(43) Pub. Date: May 2, 2024

(54) MEDICAMENT DELIVERY SYSTEMS, DEVICES, AND METHODS

(71) Applicant: Lineage Biomedical, Inc., Stuart, FL (US)

(72) Inventors: Bradley Pliskow, Gainesville, FL (US); Kevin Keller, Greenville, SC (US); Howard Preissman, Stuart, FL (US); Garrett Keller, Charleston, SC (US)

(21) Appl. No.: 18/384,765

(22) Filed: Oct. 27, 2023

Related U.S. Application Data
(60) Provisional application No. 63/420,480, filed on Oct. 28, 2022.

(51) Int. Cl. A61M 5/31; A61J 1/20

(52) U.S. Cl. CPC A61M 5/3129; A61J 1/20

(57) ABSTRACT

Medicament delivery system and methods are disclosed.

### CROSS-REFERENCE TO RELATED APPLICATIONS

The present application claims priority to U.S. Provisional Patent Application No. 63/420,480, filed Oct. 28, 2022, which is incorporated herein by reference in its entirety.

### FIELD

The present subject matter broadly relates to systems, devices, and methods for medicament delivery. In particular, disclosed herein are various embodiments of medicament injection systems, devices, and methods directed to help reduce medicament waste and improve administration precision.

### BACKGROUND

Medicament delivery systems are used for a variety of procedures and with a variety of medicaments. Some clinical applications include the administration of vaccines, anesthetics, neurotoxins, and the like. Syringes are a type of clinical medicament delivery system that generally include an outer housing, inner plunger, and often times one or more needles to inject the loaded medicament to the target site of a patient, and sometimes also to load the syringe with medicament. For example, a needle to load the syringe with medicament may be necessary if the syringe is not pre-filled and instead requires medicament transfer from a separate reservoir to the syringe. Medicament preparation (dilution, reconstitution, etc.) may also occur in the separate reservoir. But current delivery systems are challenged by various issues, including medicament waste caused by various components of the system, loss of needle lubricity, contamination and/or loss of sterility, imprecise delivery, complicated syringes that increase clinician distraction, and multiple needle exchanges to load and inject.

As an example, several issues can arise at the first stage of a medicament delivery procedure in which the medicament is prepared in and/or otherwise transferred from a separate reservoir to an injection syringe. The preparation of expensive neurotoxin such as botulinum toxin is used to illustrate some of the challenges with the current delivery systems and methods. Botulinum toxin is an expensive medicament and preparation is typically performed using one of three methods in current practice, but each has drawbacks for the clinician administering the botulinum toxin or the patient receiving it.

In one method, a vial access needle is required in addition to the injection needle. Vial access needles are removably attached to an injection syringe and are relatively large to facilitate the transfer of botulinum toxin from its vial to the syringe. The vial access needle is inserted into the botulinum toxin vial by piercing the vial septum, advanced to the botulinum toxin in the vial, and the syringe plunger is pulled proximally to draw an amount of botulinum toxin out of the vial and into the syringe. After transfer, the vial access needle is removed from the syringe and a second needle for the injection is attached to the syringe. Injection needles are typically smaller than the vial access needles and have lubricious outer coatings to facilitate injection and minimize patient pain.

The potential for the expensive botulinum toxin to be wasted is high in this method because of the propensity of the botulinum toxin to get trapped in various system components. Areas of these systems that trap and therefore waste medicament may be referred to as dead spaces, also commonly referred to as dead volume, i.e., the amount of volume of medicament remaining in a component of a medicament delivery system after use (after an injection when referring to a syringe and/or needle, and after liquid transfer from a vial when referring to a vial). As an example, vial waste can be significant and unpredictable because the amount of botulinum toxin waste remaining in the vial is variable and dependent on user technique and effort. One study concluded that neurotoxin waste in the vial averaged about 0.127 mL (N=50) with large variation (0.02-0.28 mL). (J Clin Aesthet Dermatol. 2014 June; 7(6):33-7.) But botulinum toxin waste can also occur in the injection needles and syringe, and is also unpredictable because the amount of waste in the syringe and needles is variable and dependent on the particular syringe and needles used. Up to about 0.04 mL of botulinum toxin can remain stuck in the dead space of the vial access needle, up to about 0.04 mL of botulinum toxin can remain stuck in the dead space of the injection needle, and up to about 0.04 mL of botulinum toxin can remain stuck in the syringe. These amounts can be compounded because up to five injection syringes are typically used for every vial of botulinum toxin, e.g., a 100 Unit of vial of botulinum toxin reconstituted with 2 mL of diluent.

A second method may be used to try to reduce the multiple needles and at least some of the botulinum toxin waste of the first method. However, while attempting to solve certain issues, other significant issues arise. In this method, a fixed-needle syringe is used and the single needle is used both to transfer botulinum toxin from its vial to the syringe and to perform the injection. Accordingly, the needle is inserted into the botulinum toxin vial by piercing the vial septum, advanced to the botulinum toxin in the vial, and the syringe plunger is pulled proximally to draw an amount of botulinum toxin out of the vial and into the syringe, and the loaded syringe with the needle is used to inject the medicament into the patient. But piercing the vial septum removes the lubricious coating from the needle and can damage coating that remains on the needle, increasing the insertion force required to push the needle into the patient's skin during injection and making it feel dull and painful to the patient.

A third method may be used but also presents its unique set of issues. Like the second method, it too is intended to reduce the number of needles and waste of the first method by using a single, fixedly-attached needle to both transfer botulinum toxin from its vial to the syringe and perform the injection. However, in an attempt to prevent the loss of needle lubricity of the second method, the vial septum is completely removed from the vial by the clinician, often with pliers, to provide direct access to the botulinum toxin without having to pierce the vial septum with the needle. Unfortunately, though, this method breaks the sterility of the vial and therefore creates risk of patient infection.

Moreover, imprecision is another issue that plagues conventional syringes. Syringes that inject imprecise delivery amounts increase procedural risk and cost. Moreover, procedural risk is increased if a clinician must visually monitor the syringe in an attempt to try to ensure that the correct amount is being incrementally injected in each site.

Attempts to overcome issues with medicament delivery systems have yet to provide a single solution that addresses all of the challenges, and oftentimes resulted in unduly complicated syringes to manufacture and/or use, sometimes adding additional, manual steps that the clinician must consider and perform during the medicament administration procedure, which can distract the clinician's attention away from the patient and procedure and increase procedural risk. Thus, there is a need for improved medicament delivery systems and methods.

This Background is provided to introduce a brief context for the Summary and Detailed Description that follow, and is not intended to be an aid in determining the scope of the claimed subject matter nor be viewed as limiting the claimed subject matter to implementations that solve any or all of the disadvantages or problems presented above.

### SUMMARY

Provided herein are example embodiments of medicament injection systems, devices, and methods of administering a medicament to a patient, and methods of making medicament injection systems. Embodiments include injection systems configured for clinical and non-clinical use, and methods of using and making medicament injection systems. Clinical applications include, but are not limited to, injection systems configured to inject medicament into a patient, where “patient” is non-limiting and includes living and non-living mammals (e.g., humans) and non-mammals. “Medicament” is non-limiting and includes drugs, vaccines, neurotoxins, and the like, e.g., in liquid form. In some exemplar embodiments disclosed herein, the injection systems include syringes, and are particularly suited and configured to inject neurotoxin, such as botulinum toxin solutions, into a patient. Botulinum toxin injections present unique challenges because botulinum toxin is expensive, and a procedure related thereto often requires multiple, precise injections in rapid, serial succession at precise injection site(s), e.g., for a cosmetic procedure. Accordingly, the disclosed injection syringes are configured to minimize clinician distraction, reduce and/or eliminate medicament waste, and precisely and consistently dispense incremented amounts of medicament.

Embodiments include medicament delivery components for use with the disclosed syringes, such as injection needles and medicament vial transfer adapters, e.g., to be used in the injection of botulinum toxin solutions into a patient. Injection system components can be configured to be fit and used together for injections, or some or all may be used separately. Various embodiments of packaged combinations are disclosed herein, including but not limited to packaged combinations of one or more of the following: one or more syringes, one or more needles, one or more medicament vial transfer adapters, and one or more diluent syringes (which can be pre-filled with diluent). In some embodiments, these packages can be configured for botulinum toxin procedures. For example, a package can have all necessary injection system components (with or without a reservoir of botulinum toxin) for one botulinum toxin vial for one procedure, e.g., using botulinum toxin from a single botulinum toxin reservoir for a cosmetic procedure of one patient. Components of a package can be terminally packaged together, in individual or collective sterile containers, and packaging can include one or more reservoirs of botulinum toxin (undiluted, diluted, dry, reconstituted), and/or one or more syringes can be pre-loaded with botulinum toxin, i.e., supplied to the clinician with medicament inside, where “clinician” is non-limiting to include a user of an injection system and/or user performing a method of administering botulinum toxin to a patient, e.g., to a human patient. Also disclosed herein are methods of manufacturing a medicament injection system.

This Summary is provided to introduce a selection of concepts in a simplified form that are further described below in the Detailed Description. Moreover, it is noted that the disclosure is not limited to the specific embodiments described in the Detailed Description and/or other sections of this document. Such embodiments are presented herein for illustrative purposes only. Other configurations, devices, methods, features and advantages of the subject matter described herein will be or will become apparent to one with skill in the art upon examination of the following figures and Detailed Description. It is intended that all such additional configurations, devices, methods, features and advantages be included within this description, be within the scope of the subject matter described herein and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be depicted schematically rather than literally or precisely.

FIG. 1A shows a perspective view of an example embodiment of an injection system.

FIG. 1B-1 shows an exploded view of an exemplar embodiment of an injection system.

FIG. 1B-2 shows an exploded view of an exemplar embodiment of an injection system with an anti-rotation-bias assembly.

FIG. 2A shows a perspective view of an exemplar embodiment of an injection system in a free operational state with the plunger pulled back.

FIG. 2B shows a plan view of an exemplar embodiment of an injection system in a free operational state with the plunger pulled back.

FIG. 2C-1 shows a side cutaway of an exemplar embodiment of an injection system in a free operational state.

FIG. 2C-2 shows a side cutaway of an exemplar embodiment of an injection system with an anti-rotation-bias assembly in a free operational state.

FIG. 3A shows a perspective view of an exemplar embodiment of an injection system in a metered operational state with the plunger pulled back.

FIG. 3B shows a plan view of an exemplar embodiment of an injection system in a metered operational state with the plunger pulled back.

FIG. 3C-1 shows a side cutaway of an exemplar embodiment of an injection system in a metered operational state.

FIG. 3C-2 shows a side cutaway of an exemplar embodiment of an injection system with an anti-rotation-bias assembly in a metered operational state.

FIG. 4A-1 is cross-sectional view taken along J-J of an exemplar embodiment of an injection system.

FIG. 4A-2 is cross-sectional view taken along J-J of an exemplar embodiment of an injection system with an anti-rotation-bias assembly.

FIG. 5A-1 is a cross-sectional view taken along L-L of an exemplar embodiment of an injection system.

FIG. 5A-2 is a cross-sectional view taken along L-L of an exemplar embodiment of an injection system with an anti-rotation-bias assembly.

FIGS. 5B-5F are close-up views of an exemplar embodiment of an injection system, illustrating the progression of the free end of a spring clip in operable relation to teeth of a plunger when the plunger is activated to dispense an incremented volume of medicament from the syringe.

FIGS. 5G-5K are close-up views of an exemplar embodiment of an injection system with an anti-rotation-bias assembly, illustrating the progression of the free end of a spring clip in operable relation to teeth of a plunger when the plunger is activated to dispense an incremented volume of medicament from the syringe.

FIG. 6A shows an example bias member in the form of a pin in operable relation to a plunger, and FIG. 6B shows an example bias member in the form of a spring clip and shows the angle and directional movement of the spring clip relative to a plunger.

FIG. 6C shows an example an anti-rotation-bias assembly comprising a spring clip in operable relation to a plunger, FIG. 6D shows the spring clip of the anti-rotation-bias assembly depicted in FIG. 6C, 6E shows a perspective view of the spring clip of the anti-rotation-bias assembly depicted in FIGS. 6C and 6D, FIGS. 6F and 6G illustrate side perspective views of the spring clip of the anti-rotation-bias assembly depicted in FIGS. 6C-6E, wherein FIGS. 6F and 6G illustrate the spring clip in a deflected state, and FIG. 6H shows an enlarged, partial view of the anti-rotation-bias assembly-plunger contact area of an example cruciform-shaped plunger.

FIG. 7A shows an example bias member in the form of an S-shaped spring clip in operable relation to a plunger and shows the angle and directional movement of the spring clip relative to the plunger, FIG. 7B shows the spring clip of FIG. 7A, FIG. 7C shows a perspective view of the S-shaped spring clip of FIGS. 7A and 7B, FIGS. 7D and 7E show the results of a stress test of spring clip 430 made of polycarbonate and the deflection, respectively, and FIG. 7F shows an enlarged, partial view of the spring clip-plunger contact area of an example diamond-shaped plunger.

FIG. 8A shows an example embodiment of a four-faceted plunger, FIG. 8B shows a perspective view of the plunger, FIG. 8C shows a syringe with the plunger and part of the grip removed, and FIG. 8D shows a syringe with the plunger and part of the grip removed, wherein the injection system comprises an anti-rotation-bias assembly.

FIG. 9A-1 shows an exemplar embodiment on an interior of a grip for an injection system.

FIG. 9A-2 shows an exemplar embodiment on an interior of a grip for an injection system.

FIG. 10A-1 is a cross sectional view of a pair of cooperating ribs of an example anti-rotation mechanism taken along N-N, FIG. 10A-2 is a cross sectional view of the grip depicted in FIG. 9A-2 taken along M-M, FIG. 10B-1 shows two pairs of cooperating ribs holding a four-faceted plunger in the shape of a cruciform, and FIG. 10B-2 shows two pairs of cooperating ribs formed on leg portions of an anti-rotation mechanism of an anti-rotation-bias assembly holding a four-faceted plunger in the shape of a cruciform.

FIG. 11A-1 shows a perspective view of an example embodiment of a four-faceted cruciform plunger, illustrating an enlarged, partial view of the spring clip-plunger contact area, FIG. 11A-2 shows a perspective view of an example embodiment of a four-faceted cruciform plunger, illustrating an enlarged, partial view of the anti-rotation-bias assembly-plunger contact area, FIG. 11B-1 shows a perspective view of the four-faced cruciform plunger and spring clip-plunger contact area depicted in FIG. 11A-1, and FIG. 11B-2 shows a perspective view of the four-faced cruciform plunger and anti-rotation-bias assembly-plunger contact area depicted in FIG. 11A-2.

FIG. 12 shows a perspective view of an example embodiment of a three-faceted trilobal plunger.

FIG. 13 shows an example embodiment of a near-zero dead space needle assembly configured to be used with disclosed syringes, with or without a needle shield.

FIGS. 14A and 14B show an example embodiment of a near-zero dead space vial adapter assembly configured to be used with disclosed syringes, showing the adapter cap separated from the adapter body and connected to the adapter body, respectively.

FIG. 15 shows an example embodiment of an injection kit.

FIG. 16 shows the change of operating mode of a syringe of Example 1, and example syringe asymmetry that visually and tactilely indicates a selected operating mode.

### DETAILED DESCRIPTION

Various example embodiments are shown in the figures and further described below. Reference is made to these examples in a non-limiting sense, as it should be noted that they are provided to illustrate more broadly applicable aspects of the devices, systems and/or methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present disclosure. All such modifications are intended to be within the scope of the claims that can be made herein.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

Where a range of values is provided, it is understood that every intervening value, between the upper and lower limit of that range and any other stated or intervening value in the stated range is encompassed within the embodiments described herein. Also, it is contemplated that any optional feature of the inventive variations described may be set forth and claimed independently, or in combination with any one or more of the features described herein. Moreover, no limitations from the specification are intended to be read into any claims, unless those limitations are expressly included in the claims.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

The subject matter described herein and in the accompanying figures is done so with sufficient detail and clarity to permit the inclusion of claims, at any time, in means-plus-function format pursuant to 35 U.S.C. Section 112, Part (f). However, a claim is to be interpreted as invoking this means-plus-function format only if the phrase “means for” is explicitly recited in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, acts, steps, or elements of the embodiments may be recited in or added to the claims, as well as negative limitations that define the inventive scope of the claims by features, functions, acts, steps, or elements that are not within that scope.

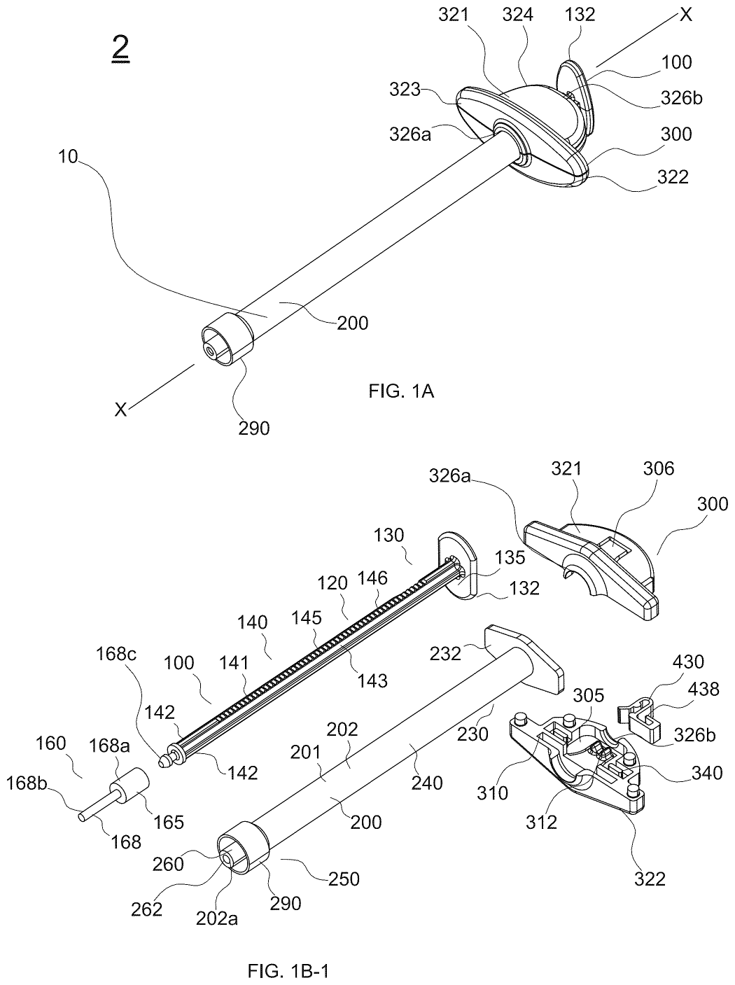

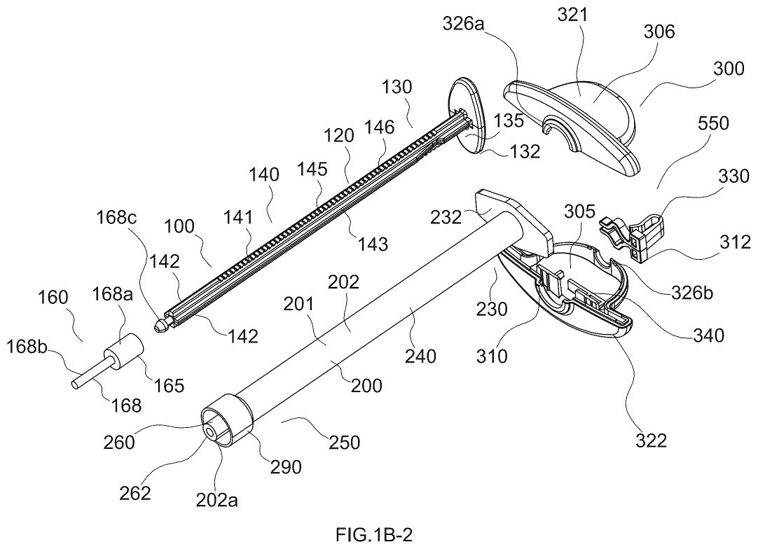

FIGS. 1A to 3C-3 show example embodiments of a medicament injection system 2. Injection system 2 includes syringe 10 that has barrel portion 200 and plunger portion 100 that is sized and dimensioned to be at least partially received within barrel portion 200. Syringe defines central axis X-X. Syringe 10 includes at least two operating states, and can be configured to operate with audible and/or tactile feedback. Feedback embodiments can be configured to operate with audible and/or tactile feedback in one state, and different (including less intensity) or no audible and/or tactile feedback in another state. In other words, the relative intensities of audible and/or tactile feedback can provide operational state information to the clinician, and/or the presence and absence of audible and/or tactile feedback can provide operational state information to the clinician. The syringe 10 is easily switchable from one operating state to another just by manipulation of the plunger portion 100 (by applying at least a threshold amount of twisting force thereto), and the selected operating state is quickly and easily visually and/or or tactilely identifiable. The selected operating state is maintained by the syringe 10 itself, without any additional action or effort by the clinician, until intentionally switched to another operating state by the clinician.

Syringe 10 operates by manual manipulation (e.g., hand manipulation) of plunger portion 100 relative to barrel portion 200. For example, plunger portion 100 concentrically resides within an interior space of barrel portion 200 and is configured to axially translate relative to the barrel portion 200 when the two are operably connected and, upon application of manual force to a proximal portion of the plunger portion 100. Manual force includes a pulling force to load medicament into the barrel portion 200 from a medicament vial and a pushing force to incrementally dose the medicament from the barrel portion 200. Loading can be needle-less loading in that a needle-free syringe can be used to transfer medicament to the syringe 10 from a vial. A needle-free syringe for loading preserves the vial integrity and sterility of remaining medicant in the vial after a portion of medicament has been withdrawn into the syringe, and reduces the number of needles for a procedure. Embodiments include syringes 10 with needles, e.g., for injections, and a needle can be luer-lock connected to the syringe 10 for incremented dosing. Syringes 10 can be any suitable size, e.g., may be 0.5 mL, 1 mL, 2 mL, or larger or smaller volume syringes.

Embodiments include injection systems 2 in which at least one component has zero or near-zero dead space and, therefore, the disclosed injection systems 2 (and/or components thereof) are referred to as having zero or near-zero dead space. For example, a syringe 10 can have a zero or near-zero dead space distal tip such that all or substantially all (also referred to as nearly all) of the medicament can be dispensable from the syringe 10. Embodiments include a needle having a near-zero dead space hub such that substantially all (also referred to as nearly all) of medicament can be dispensable from the needle when attached to a syringe 10, and/or a vial adapter can have near-zero dead space such that substantially all (also referred to as nearly all) of the medicament can be withdrawn through the adapter when used with a syringe 10. Some or all of the injection system 2 components described herein can be used separately or together to provide injection systems 2 with no (zero) or near-zero dead space.

In additional to axial translation relative to the barrel portion 200, plunger portion 100 can be configured to rotate relative to barrel portion 200 360-degrees about central axis X-X (see, e.g., FIG. 1A), and can be selectively locked and unlocked to prevent and permit, respectively, rotational movement within the barrel portion 200. For example, syringe 10 can be configured to hold plunger portion 100 in an anti-rotation orientation relative to the barrel portion 200 such that plunger portion's 100 translational motion is permitted but rotational motion is prevented. Further, the barrel portion 200 can selectively release the hold on the plunger portion 100 to permit rotation.

Rotational motion of plunger portion 100 relative to barrel portion 200 when the two are operably connected can be enabled upon application of sufficient torque to the plunger portion 100 to overcome anti-rotational resistance applied to the plunger portion 100, thereby unlocking the plunger portion 100 for rotation. In this manner, the syringe 10 can be lockable at a selected plunger portion 100 operating state of a metered operational state and a free operational state, preventing unintentional rotation to an unintended state. As described herein, “free operational state” and “metered operational state” of a syringe 10 refer to selectable operational states of a syringe 10 defined by whether a “free facet” or “rack facet” of a plunger portion 100 of the syringe 10 is in operable, direct contact with a free end of a bias member of the syringe 10. As described herein, “rack facet” includes a plunger facet having a rack of metering teeth, and “free facet” includes a plunger facet that does not include a rack of metering teeth, i.e., a non-rack facet. In this context, the “teeth,” “tooth,” “rack,” “rack features,” “tooth feature,” and “teeth features” can all be interchangeably used in reference to the rack of metering teeth. As described herein, teeth features can be in the form of depressions, notches, grooves, valleys, and the like, that accept a pawl, tip or point therein. Those of skill in the art will appreciate that other forms of rack facets or teeth can be utilized. Together, the elements can be regarded as an overall ratchet-tooth rack mechanism or assembly. Accordingly, a metered operational state permits incremented injection of precise amounts of medicament from the syringe 10, and a free operational state permits at least lower relative resistance axial translation of plunger portion 100, e.g., for loading the syringe 10 with medicament, due to the lack of a ratchet-tooth rack assembly. As such, plunger portion 100 is prevented from rotating, and therefore is set to a given operational state, by the anti-rotational mechanism 312, and therefore no additional action or effort by the clinician is required to maintain the selected operational state.

As best shown in FIG. 1B-1, barrel portion 200 has a first end that is the proximal end 230, a second end that is the distal end 250, a medial cylinder 240 that has an outer surface 201 and consistent inner diameter, an inner surface 202 that defines an inner circumference diameter of the barrel portion 200, and an interior cavity that receives at least a portion of plunger medial shaft 140. Barrel portion 200 cooperates with plunger portion 100 to form a medicament chamber defined by a distal end of a piston 160 of the plunger portion 100, a proximal end of a needle hub when operably connected thereto, and the barrel inner wall. In some embodiments, outer surface 201 can include a graduated volumetric measurement scale, e.g., a visual scale calibrated to 1 mL with intervening gradations. Those of skill in the art will appreciate that other visual scales and gradations can be utilized with the embodiments described herein.

In some exemplar embodiments, and as best shown in FIG. 1B-1, the barrel portion's proximal end 230 includes proximal barrel flange 232 located in the barrel flange-receiving cavity 310 of at least partially hollow housing 305 of grip 300. In some embodiments, and as will be described in further detail below, housing 305 includes at least one bias member 330, 360, or 430 that is biased towards the plunger portion 100 when in a biased state such that a free end of the bias member 330, 360, or 430 is in constant contact with the plunger portion 100. By way of example only, embodiments are primarily described herein with respect to bias member 330 and 430, and shown in figures primarily with respect to bias member 330 and 430, and the description and figures are non-limiting and it is to be understood that bias members having different configurations, including but not limited to bias member 360, are contemplated and can be used instead of or in addition to bias members 330 or 430.

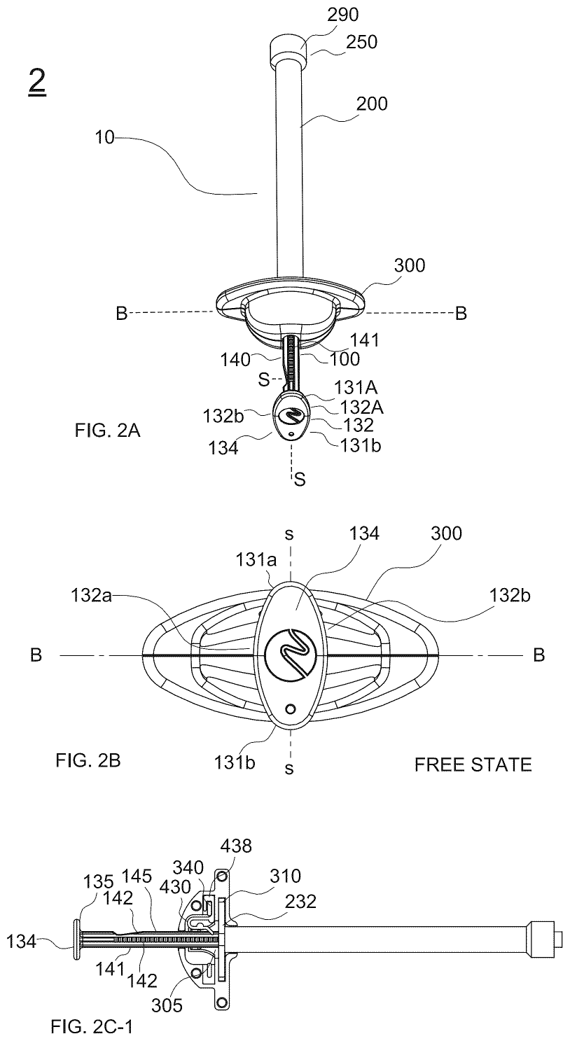

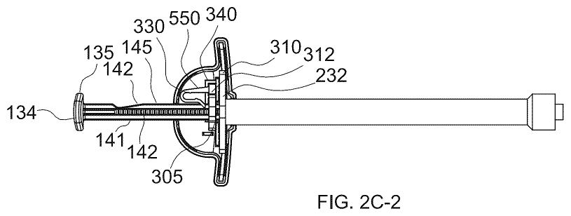

In some embodiments, and as will be described in further detail below, an anti-rotation mechanism 312 can extend from the housing 305 and is configured to secure the plunger portion 100 in a selected orientation relative to the barrel portion 200 as the bias member 330, 360, or 430 biases towards the plunger portion 100 when in the biased state. In some exemplar embodiments, and as shown in FIG. 1B-1, the anti-rotation mechanism 312 can be separate structure from the bias member 330, 360, or 430 (bias member 430 is depicted in FIG. 1B-1), and can be built into and extend from the grip 300 or a surface of housing 305 thereof. In other exemplar embodiments, and with reference to FIG. 1B-2, the anti-rotation mechanism 312 is integrated or welded together with the bias member 330, 360, or 430 (bias member 330 is shown in the exemplar embodiment depicted in FIG. 1B-2) to form an anti-rotation-bias assembly 550.

As best shown in FIGS. 1A to 1B-2, grip 300 comprises a first side 321, an opposing second side 322, a distal-facing side 323 and proximal-facing side 324. Grip 300 includes two barrel-receiving cut-away areas 326a,b configured to receive barrel portion 200. Grip 300 can include viewing window 306 to view an indication of a selected operating state of the syringe 10 (e.g., a metered state or a free state of the syringe 10).

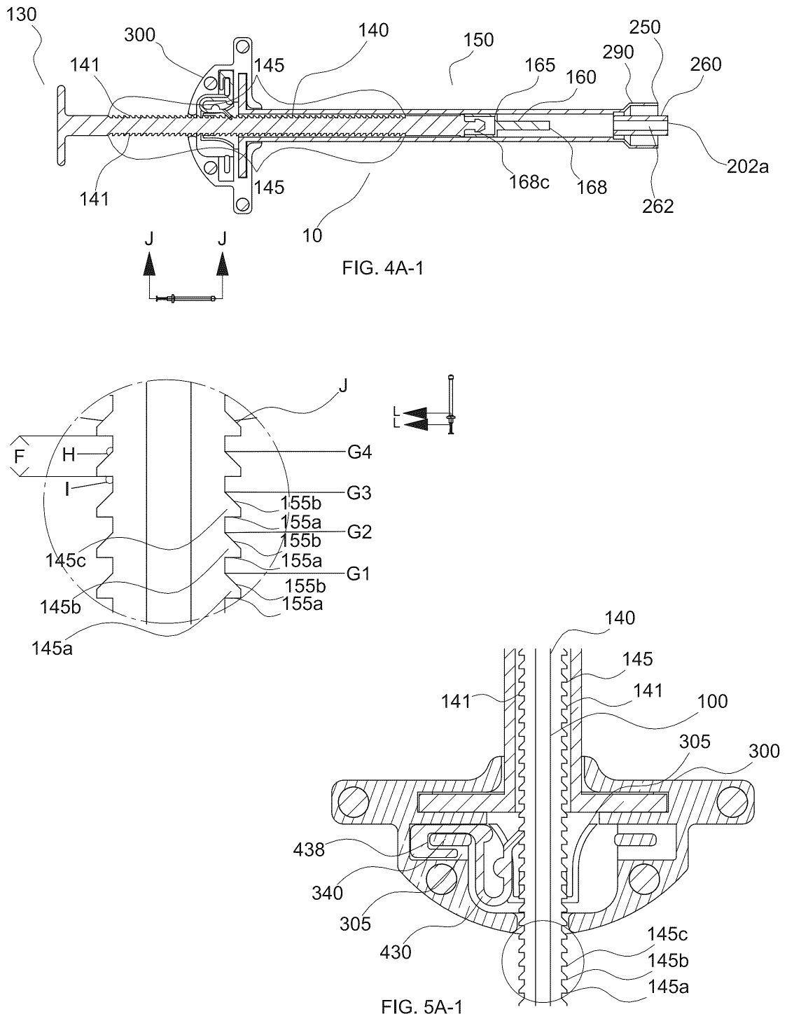

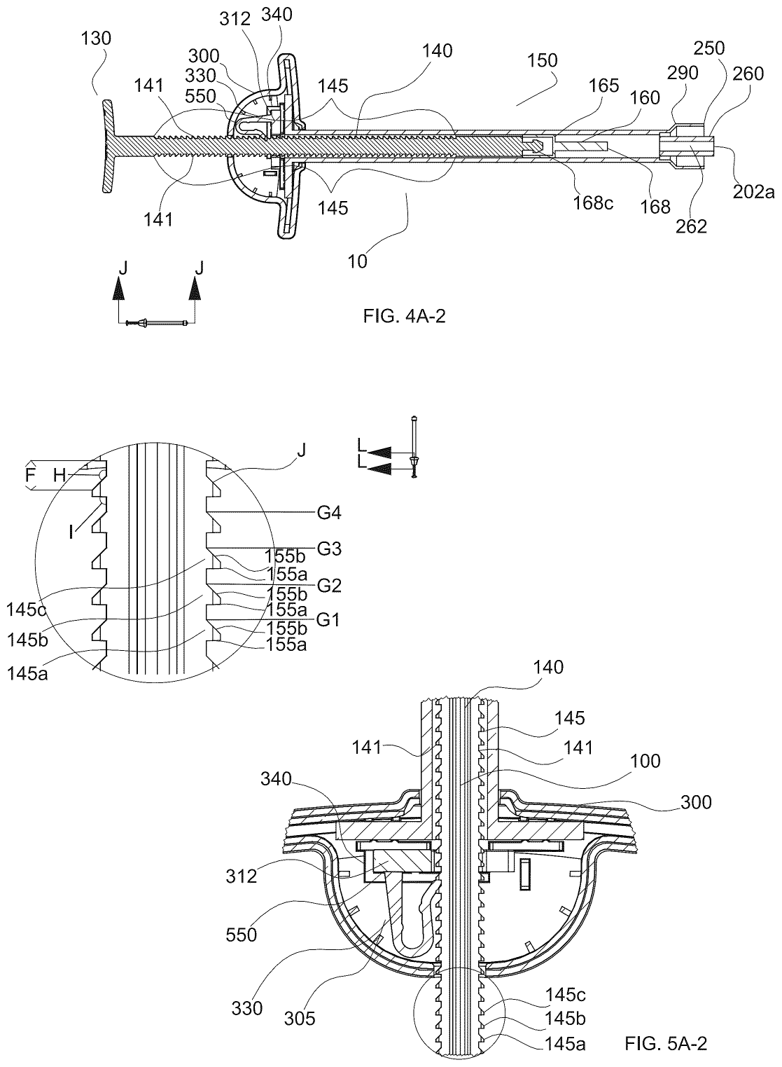

In some embodiments, and as best illustrated in FIGS. 1B-1 and 4, the barrel portion's distal end 250 includes a reduced diameter neck portion 260 having an inner surface 202a that forms space 262 sized and shaped to receive the distal tip of the plunger portion 100 that includes distal end piston 160. In some embodiments, the exterior of barrel neck portion 260 includes luer-lock interface 290. Luer lock interface 290 is configured to interface with one or more of a luer-lock interface of a needle hub of a needle and a luer-lock interface of a medicament vial adapter.

According to one aspect of the embodiments, the one or more bias members 330, 360, or 430 can automatically, upon axial translation of the plunger portion 100, cooperate with the plunger portion 100 to form a bias member-plunger assembly that easily and smoothly dispenses precise, incremented amounts of medicament from the syringe 10, easily and smoothly advances the plunger portion 100, and easily and smoothly arrests axial translation of the plunger portion 100 at each increment (i.e., it provides a strong “catch”). According to another aspect of the embodiments, the bias member-plunger assembly can also produce audible and/or tactile feedback at each increment. In some embodiments, the bias member 330, 360, or 430 never disengages from the plunger portion 100, thereby eliminating the possibility of a device failure in which feedback in a metered state is not provided due to a lack of contact between the bias member and plunger portion 100. More than one bias member 330, 360, or 430 can be used. In some embodiments, if more than one bias member 330, 360, or 430 is used, the bias members 330, 360, or 430 can be the same or different in one or more respects. By way of example only, embodiments are primarily described with respect to a single bias member 330, 360, or 430, and the description and figures are non-limiting and it is to be understood that more than one bias member 330, 360, or 430 can be used in a single syringe.

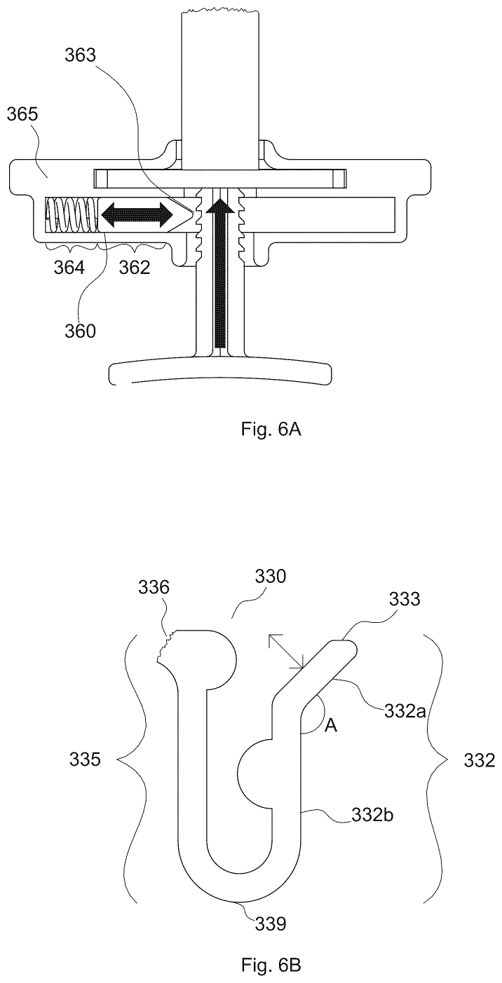

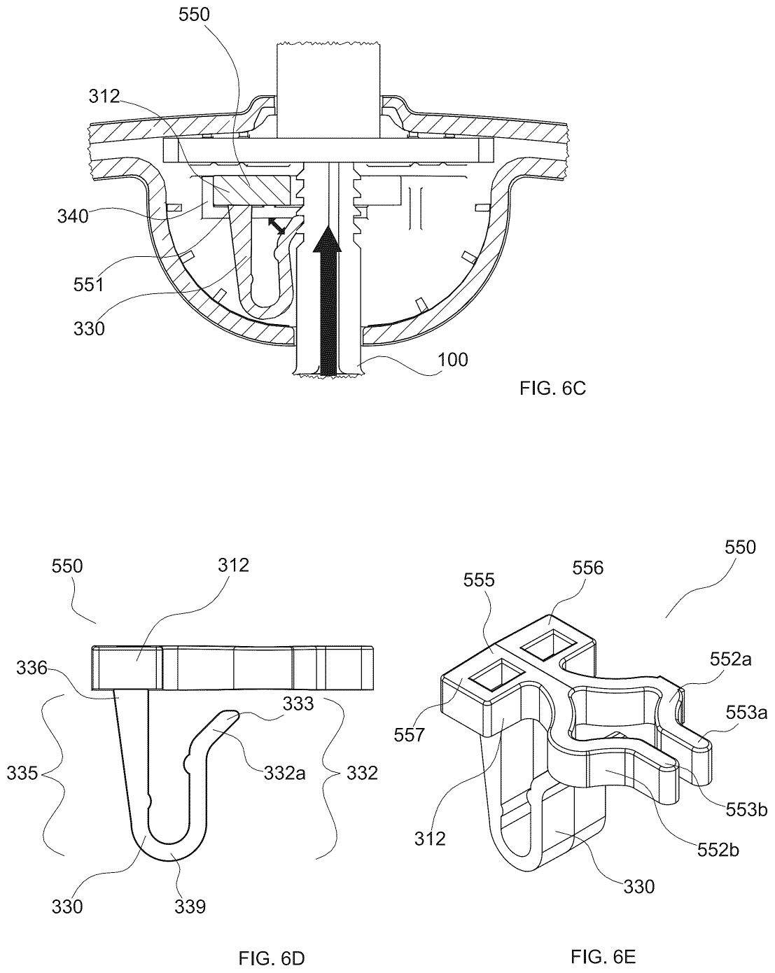

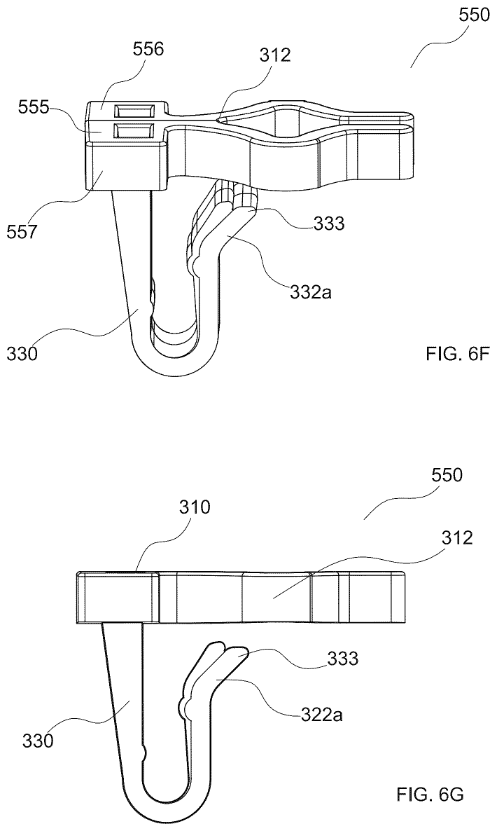

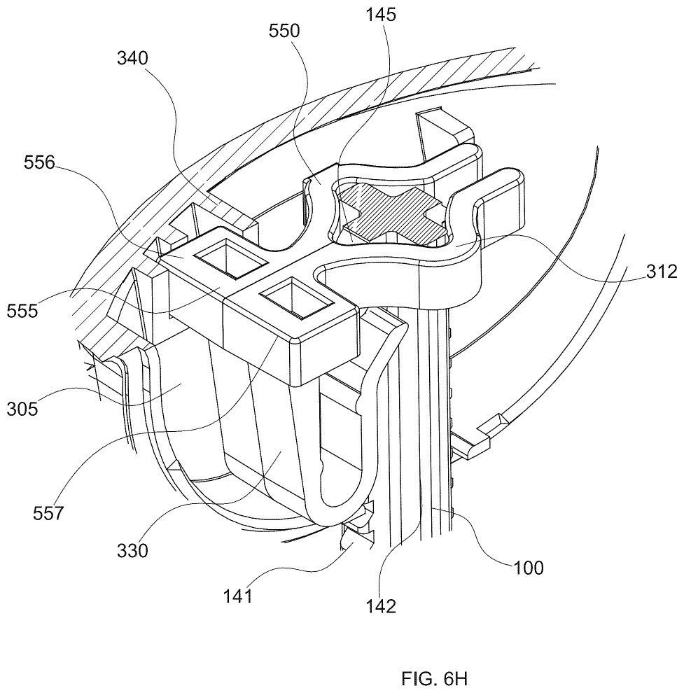

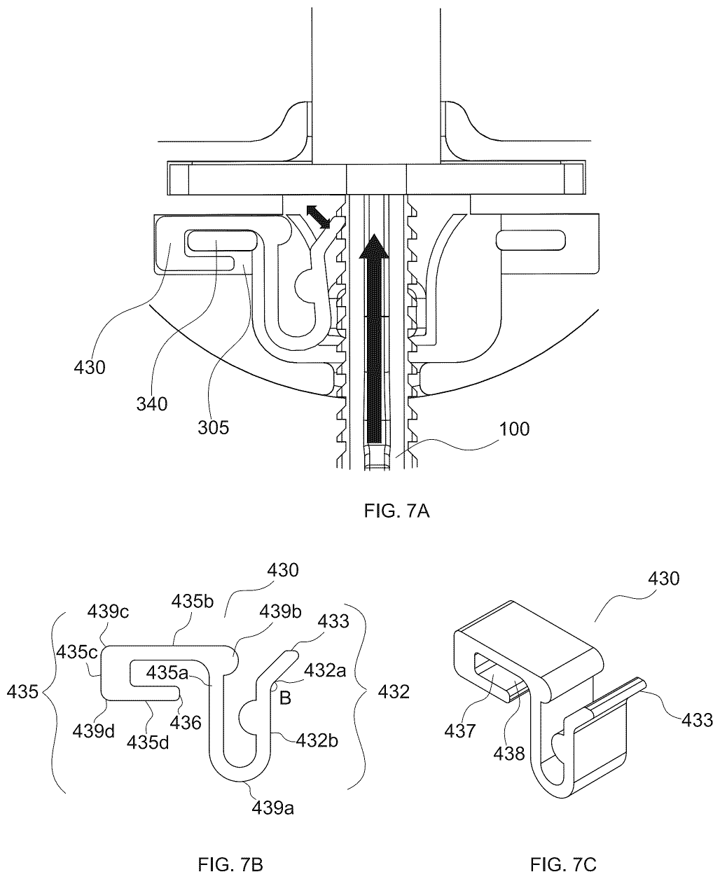

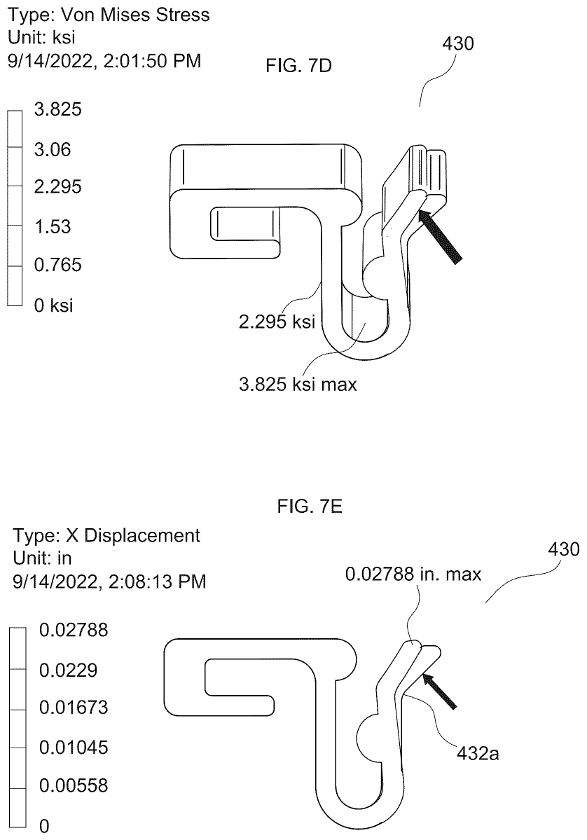

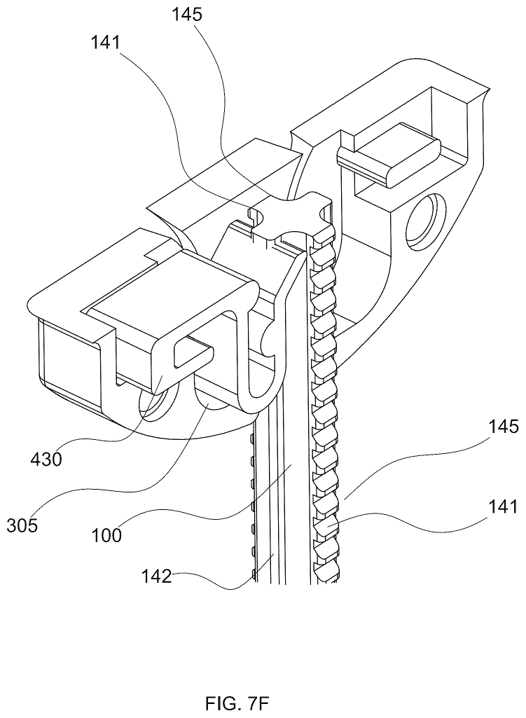

According to some embodiments, the bias member 330, 360, or 430 can be any suitable configuration, including pin-shaped (linear), S-shaped, J-shaped, and the like. Those of skill in the art will appreciate that other bias member shapes and configurations can be utilized with the embodiments described herein. As described in example embodiments herein, a bias member 330, 360, or 430 can be in the form of a pin (bias member 360, see, e.g., pin of FIG. 6A), or a spring clip (bias member 330 or 430, see e.g., S-shaped spring clips of FIGS. 6B-7F). As such, embodiments of the bias member 330, 360, or 430 described herein can also be referred to as the pin 360 or the spring clip 330 or 430. In some exemplar embodiments, bias member 330 (in some embodiments, bias member 330 of anti-rotation-bias assembly 550) or 430 can be retained within hollow housing 305 at least partially by a retention feature 340 (see, e.g., FIGS. 1B-1, 1B-2, 2C-1, 2C-2, 3C-1, 3C-2, 5A-1, 5A-2, 6C, 6H, 7A, 7F, 8C, 11A-1, and 11A-2). In some embodiments, the bias member, 330 or 430 and retention feature 340 are configured so that the retention features 340 hold one end (fixed end) of the bias member 330 or 430 in place to prevent it from translation within hollow housing 305, but permit another end (free end) of the bias member 330 or 430 to freely, and continuously without disengagement, translate in position within hollow housing 305. In some embodiments, retention feature 340 is U-shaped so as to partially surround an outer surface of the anti-rotation-bias member assembly 550.

The free end of the bias member 330 or 430 slidingly travels over a facet surface of the plunger portion 100 to which it is contacted when the plunger portion 100 is axially translated, where the at least one facet is a free facet or rack facet, depending on the selected operational state of the syringe 10. As described herein, at least a portion of the outer surface of the plunger portion 100 includes a rack of a plurality of metering teeth that are each spaced apart a distance that defines the incremented injection volume and cooperate with the bias member 330 or 430 free end to increment injections. Accordingly, a syringe 10 that is set to increment injections is positioned with a rack facet of the plunger portion 100 in contact with the bias member 330 or 430 free end, and is in a metered state, sometimes also referred to as an inject state, when the plunger portion 100 is pulled back relative to the barrel portion 200 and readied for injections. In some embodiments, as the spring clip 330 or 430 free end travels over a tooth of the plunger portion 100 during axial translation of the plunger portion 100 in a distal direction within barrel portion 200 when the syringe 10 is in a metered state, the bias member 330 or 430 free end is completely free to travel to impact a subsequent tooth of the rack. Pushing force is applied to the plunger portion 100 to overcome tooth resistance and advance the free end of the bias member 330 or 430 to another tooth and stop or be caught until additional force is applied. In this manner, the bias member 330 or 430 cooperates with successive teeth of the plunger portion 100 to enable precise, controlled, incremented medicament injections. In some feedback embodiments, this provides tactile feedback to the clinician of plunger portion 100 advancement and the amount injected because distances between each tooth correspond to known injected amounts, which correspond to the scale of the syringe. Tactile feedback also confirms that the syringe 10 is in a metered operational state. Accordingly, embodiments include syringes 10 that are configured to provide tactile feedback that corresponds to each incremented injection amount, as well as embodiments that do not provide tactile feedback that corresponds to each incremented injection amounts.

In some embodiments, a bias member 330 (in some embodiments, bias member 330 of anti-rotation-bias assembly 550), 360, or 430 engages with a rack portion of a plunger portion 100 and creates an audible snap sound each time the bias member 360, 330, or 430 free end passes over a tooth of the rack. An audible snap can serve as audible feedback to the clinician of plunger portion 100 advancement. Likewise, lack of an audible snap during axial translation may confirm that the bias member 360, 330, or 430 free end is in engagement with a non-rack portion of the plunger portion 100, and is therefore in a free state (sometimes referred to as withdrawal state) rather than a metered state. The metered injections, i.e., each “snap”, can correspond to gradations of a measurement scale of the outer surface of the barrel portion 200. Accordingly, embodiments include syringes 10 that are configured to provide audible feedback that corresponds to each incremented injection amount, as well as embodiments that do not provide audible feedback that corresponds to each incremented injection amount.

Embodiments described herein can include syringes 10 that are configured to provide one or both of an audible and/or tactile feedback that correspond to each incremented injection amount, as well as embodiments that do not provide one or both of an audible and/or tactile feedback that correspond to each incremented injection amount.

A bias member in the form of bias pin 360 is shown in FIG. 6A. In some exemplar embodiments, and as shown in FIG. 6A, bias pin 360 includes first portion 362 in the form of a pin head having a first end 363 that is a plunger-contacting end (also referred to as the free end), and a second portion 364 in the form of a spring having a second end 365 that is a fixed end held in place, wherein the fixed end does not contact the plunger portion 100. In some pin embodiments, the bias member 360 moves in a direction perpendicular to the axial translation motion of the plunger portion 100. Accordingly, as shown by the arrows, the force applied to advance the plunger portion 100 is translated by 90 degrees to move the pin 360 and initiate motion, and once motion of the plunger portion 100 is initiated, the pin force is at 90 degrees relative to the plunger portion's 100 motion.

Effective bias member-plunger operation is highly dependent upon the depth/angle of the plunger teeth and friction between mating surfaces. For example, shallow, low angle teeth on the plunger portion 100 result in easy/smooth advancement of the plunger portion 100, but less ability of the bias member 360, 330, or 430 free end to “catch” the plunger portion 100 and arrest motion. In these embodiments, however, advancement can be smoother. In some embodiments, deep, high angle teeth on the plunger portion 100 result in stronger “catch,” but also unsmooth advancement of the plunger portion 100. Therefore, spacing between respective teeth that is very small (e.g., embodiments that include spacing between respective teeth of about 0.02 inches to about 0.1 inches, e.g., about 0.045 inches, such as some 1 mL syringe embodiments) can result in a higher likelihood that the bias member 360, 330, or 430 free end will not arrest motion at each increment once plunger portion 100 motion is initiated. In other words, the likelihood that a clinician will accidentally skip increments during injection is increased.

FIGS. 6B to 7F show example embodiments of bias members 330 (in some embodiments, bias member 330 of anti-rotation-bias assembly 550) or 430 in which the free end of the bias member 330 or 430 moves at about a 45-degree angle to the axial translation motion of the plunger portion 100 (see, e.g., arrows of FIGS. 6B, 6C, and 7A). Accordingly, the force applied to advance the plunger portion 100 must only be translated by about 45 degrees in order to move the spring clip 330 or 430 and initiate motion. Once motion of the plunger portion 100 is initiated, a component of the spring clip 330 or 430 force directly opposes motion. This component of force that opposes motion makes it easier to arrest motion once motion is initiated. This also makes the operation less influenced by friction between mating surfaces. The about 45-degree angle provides an equal balance between ease of advancing the plunger portion 100 and ease of arresting motion at each increment and, therefore, reduces the likelihood that a clinician will accidentally skip increments during injection.

The embodiments of FIGS. 6B to 7F have one or more linear segments connected by transitions, and are shown in the form of spring clips 330, 430. In some embodiments, spring clips 330, 430 can be made of metal or non-metal such as a polymer, wherein materials include but are not limited to stainless steel, polycarbonate, ABS, acetal, or polypropylene. Those of skill in the art will appreciate that other suitable materials for the spring clips can be utilized with the embodiments described herein.

In some exemplar embodiments, and as depicted in FIGS. 6B-6H, spring clip 330 (in some embodiments, spring clip 330 of anti-rotation-bias assembly 550) comprises a first portion 332 having first end 333 that is the plunger-contacting end (free end). Further, in some embodiments, spring clip 330 can also comprise a second portion 335 having a second end 336 that is the fixed end (held end or non-plunger-contacting end), and transition portion 339 in the form of a “U” shape therebetween. First end 332 includes first section 332a and second section 332b forming angle A. In some exemplar embodiments, angle A can be between about 120 degrees and about 150 degrees(e.g., can be about 135 degrees in some embodiments). Those of skill in the art will appreciate that other angles and configurations of the spring clips and components thereof can be utilized with the embodiments described herein. Spring clip 330 primarily strains in transition area 339 when in a biased state. As described herein, some embodiments include a free end 333 that moves at about a 45-degree angle to the motion of the plunger portion 100 (axial translational motion) to which it is contacted.

In some embodiments, and with particular reference to FIG. 6C-6H, the spring clip 330 can be part of the anti-rotation-bias assembly 550, and as such, is integrated with the anti-rotation mechanism 312. As shown in FIGS. 6C-6H, the anti-rotation mechanism of the anti-rotation-bias assembly 550 can be in the form of a cantilever extending from a top surface 551 of the spring clip 330. In some embodiments, the anti-rotation mechanism 312 can be integrated with and extend from the second end 336. In some embodiments, and as best shown in FIGS. 6E, 6F, and 6H, the anti-rotation mechanism 312 of the anti-rotation-bias assembly 550 comprises one or more leg portions 552a, b. In some exemplar embodiments, the one or more leg portions 552a,b include a first leg portion 552a and a second leg portion 552b, wherein each leg portion 552a,b forms a pair of the one or more gripping ribs 313. Specifically, the first leg portion 552a can form gripping ribs 313a and 313b, and the second leg portion 552b can form gripping ribs 313c and 313d. In this manner, the first leg portion 552a extends radially from the top surface 551 to a first foot 553a and the second leg portion 552b extend radially from the top surface 551 to a second foot 553b such that a channel 554 (also herein referred to as a window 554) is formed between the leg portions 552a,b and the feet 553a,b, wherein the window 554 is sized and configured for receipt of the plunger portion 100. Specifically, the first leg portion 552a and the second leg portion 552b extend radially from the top surface 551 such that the gripping ribs 313a, b and gripping ribs 313c, d are positioned across from one another so as to form the one or more gripping cut-outs 314a, b (best shown in FIGS. 6E, 6F, and 6H) the define the window 554. According to some embodiments, the first leg portion 552a and the second leg portion 552b are connected together by a bridge portion 555. In some embodiments, the bridge portion 555 is welded together or otherwise integrated with the top surface 551 of the bias member 330. In some embodiments, the anti-rotation-bias assembly 550 is secured in place in the housing 305 by a first side 556 of the bridge portion 555 being disposed in one or more apertures that extend from an interior surface of the housing 305, and a second end 557 of the bridge portion 555 being disposed in one or more apertures that extends from an opposing interior surface of the housing 305. Specifically, in some embodiments, the retention feature 340 extends from the interior surface of the housing 305. In some embodiments, the one or more apertures extending from the interior surface of the housing 305 define the retention feature 340. Specifically, in some embodiments, the retention feature 340 forms a wall extending from the interior surface of housing 305 (FIGS. 1B-2, 2C-2, 3C-2), wherein the wall is configured to surround or at least partially surround a portion of the anti-rotation-bias assembly 550 so as to secure it in place in the housing 305.

According to another aspect of the embodiments, and as best shown in FIGS. 6C and 6H, the anti-rotation-bias assembly 550 is disposed within the housing 305 of grip 300, and oriented such that (1) the anti-rotation mechanism 312 portion of the anti-rotation-bias assembly 550 interfaces with the plunger portion 100, and (2) the first end 333 of the bias member 330 can slidingly travel over the facet surface (free facet 142 or rack facet 141, depending on the operational state of the syringe 10, in FIG. 6H, bias member 330 is slidingly traveling over rack facet 141) of plunger portion 100. Specifically, and as best shown in FIG. 6H, in some embodiments, plunger portion 100 is movably positioned with the channel or the cut-out portion 314a,b of the anti-rotation mechanism 312 portion of the anti-rotation-bias assembly 550. More specifically, in some embodiments, the anti-rotation mechanism 312 portion of the anti-rotation-bias assembly 550 is positioned such that the first leg portion 552a and the second leg portion 552b extend perpendicularly or substantially perpendicularly relative to the plunger portion 100 disposed therebetween. Even more specifically, the configuration of the anti-rotation-bias assembly 550 provides the window 554 through which the plunger portion can movably and axially be disposed in. Further, in some embodiments, and as best depicted in FIGS. 6C, 6F and 6G, the first end 333 of the bias member 330 of the anti-rotation-bias assembly 550 is configured to move at about a 45-degree angle to the axial translation motion of the plunger portion 100.

Specifically, FIGS. 6F and 6G shows the deflection of spring clip 330 of anti-rotation-bias assembly 550 under load.

The anti-rotation-bias member assembly 550 offers several design advantages. For example, in embodiments wherein the anti-rotation mechanism 312 and the spring clip 330 are not in the same position relative to one another at a same time, the amount of pulling force and/or pushing force required can vary. Because the anti-rotation mechanism 312 is integrated with the spring clip 330 in the anti-rotation-bias assembly 550, the anti-rotation mechanism 312 and the spring clip 330 are always positioned in the same configuration relative to one another. As such, the amount of pulling force and/or pushing force will remain consistent, thereby improving dose accuracy and making the injection system 2 less prone to error. Additionally, the anti-rotation-bias assembly 550 allows for smoother transitions between operational modes. For example, when the clinician is switching from one operating state to another by manipulation of the plunger portion 100 (by applying at least a threshold amount of twisting force thereto), the anti-rotation-bias assembly 550 results in easy/smooth rotation of the plunger portion 100. In this manner, the likelihood that the plunger portion 100 is stuck in between operational modes is reduced.

In some embodiments, and as shown in FIGS. 7A-7F, spring clip 430 comprises a first portion 432, a second portion 435, and a “U”-shaped transition 439a therebetween. As best shown in FIG. 7B, first portion 432 can comprise first section 432a and second section 432b forming angle B, and free end 433. In some embodiments, angle B can be between about 120 degrees and about 150 degrees, e.g., can be about 135 degrees. Those of skill in the art will appreciate that other angles and configurations of the spring clips and components thereof can be utilized with the embodiments described herein. Second portion 435 comprises first section 435a, second section 435b, third section 435c, and fourth section 435d, transitions 439b,c,d, and second end 436 (best shown in FIG. 7B). As best illustrated in FIG. 7B, first section 435a and transition 439a are two primary areas of strain of spring clip 430. The two primary strain areas increase audibility and tactility, compared to a bias member 330 that strains in only one primary area, such as a simple cantilever. Therefore, spring clip 430 provides enhanced feedback to the clinician as compared to a spring clip that only strains in one primary area (e.g. a simple cantilever), for embodiments that are configured to include audible and/or tactile feedback. As noted, spring clips described herein can comprise any suitable shape and form, including spring clips having more than two areas of strain, e.g., S-shaped spring clips having more than two areas of strain.

Compared to a simple cantilever design, spring clip 430 offers several design advantages. In a simple cantilever design, deflection of the free end results in high stress/strain concentrated in one area, at the base of the cantilever. However, spring clip 430 is “U” shaped such that the force required to deflect the free end 433 results in strain in two primary areas, 435a and 439a. The “U” shape reduces the strain of the spring clip 430 relative to a simple cantilever of a similar size footprint. This allows spring clip 430 to be a small part suitable for a small (e.g., 1 mL) syringe 10. In addition, due to the “U” shape of spring clip 430, the distribution of forces results in low enough stresses in the spring clip 430 material that a spring clip 430 made of plastic can be used (instead of a metal material, for example) without concern for failure due to yield or failure due to creep/stress relaxation. Accordingly, spring clip 430 can be made of polycarbonate, and the like. Those of skill in the art will appreciate that other suitable materials can be utilized for spring clip 430.

Still referring to FIGS. 7A-7F, upon force applied to first portion 432 of spring clip 430 by the plunger portion 100 and,—specifically, a force applied perpendicular to the surface of 432a, the free end 433 of the spring clip 430 deflects away from the plunger portion 100 (best shown by two-headed arrow of FIG. 7A). In some embodiments, spring clip 430 is configured to deflect about 0.010 inch to about 0.06 inch, when about 0.1 to about 1.0 lb-force (in some embodiments, up to about 0.6 lb-force) is applied to the spring clip 430 by the plunger portion 100.

FIG. 7D shows a Von Mises stress analysis of spring clip 430 made of polycarbonate and shows the stress distribution of polycarbonate spring clip 430 under load. FIG. 7E shows the deflection of the spring clip 430 in inches. Application of about 0.315 lb-force to the spring clip 430, as shown by the arrow (perpendicular to the spring clip surface 432a) deflects the free end of the spring clip 430 by about 0.035 inches. In some embodiments, the component of spring clip 430 deflection that is in the x-direction (i.e., perpendicular to plunger portion 100 motion, needed to bring the spring clip 430 out of a rack gap G) is about 0.028 inches. The resulting maximum stress in the spring clip 430 is about 3.825 ksi, which is well below the yield stress of polycarbonate.

In some embodiments, and with reference to FIGS. 6B-7F, the fixed end of a bias member (e.g. fixed end 336 of spring clip 330, or fixed end 436 of spring clip 430) can be retained by a spring clip retention feature 340. The specific retention feature 340 used can be selected at least in part by one or more of the configurations and materials of the bias member 330, 430 it is configured to retain. A retention feature 340 can be configured to retain a bias member 330 (in some embodiments, bias member 330 of anti-rotation-bias assembly 550) or 430 using, for example, a male-female connection, interference fit, glue, weld, and the like.

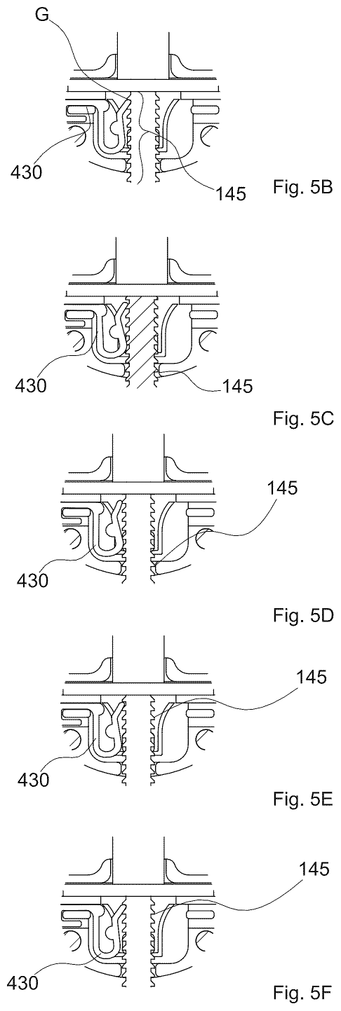

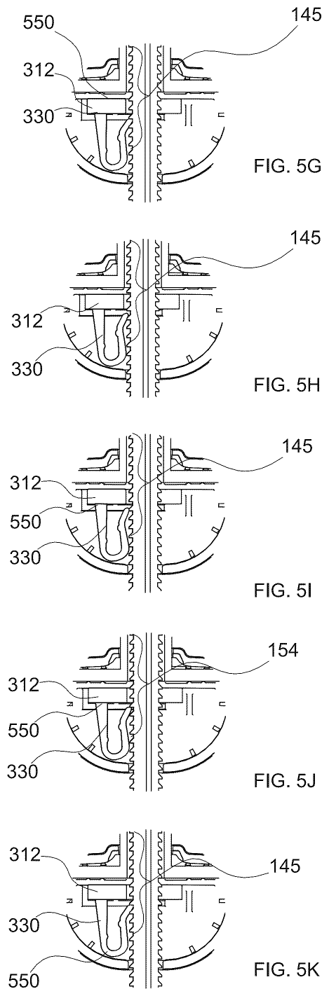

In some exemplar embodiments, and as shown in FIGS. 7A-7C, the spring clip sections and transitions of second portion 435 of spring clip 430 form retention hook 437 defining space 438. Hook 437 is configured to hook at least partially around retention feature 340 of housing 305, where retention feature 340 can be in the form of a raised protrusion from a surface of housing 305 (see, e.g., FIGS. 1B-1, 2C-1, 3C-1, 5A-1, and 7A) that at least partially occupies space 438 when retaining the spring clip 430. In other embodiments, and as shown in FIG. 5A-2, the retention feature 340 can extend from a surface of housing 305 and at least partially surrounds the anti-rotation-bias member assembly 550 and extends across a bottom surface of the anti-rotation mechanism 312 extending therefrom so as to securely retain the bias member 330 in position within hollow housing 305.

In some embodiments, housing 305 can also include plunger anti-rotation mechanism 312 (see, e.g., FIGS. 1B-1, 1B-2, 9A-1, 10A-1, 10B-1 and 10B-2). Without any additional action or effort by the clinician, anti-rotation mechanism 312 holds the plunger portion 100 in a selected orientation relative to the barrel portion 200 and resists plunger portion 100 rotation. In this regard, plunger anti-rotation mechanism 312 automatically resists rotational movement of the plunger portion 100 about central axis X-X (shown in FIG. 1A) and prevents the accidental rotation of the plunger portion 100 from an intended operating state to an unintended operating state (e.g., prevents accidental rotation of the plunger portion 100 during injection from metered state to free state). In some embodiments, the anti-rotation mechanism 312 is configured to prevent rotation of the plunger portion 100 relative to the barrel portion 200 by applying rotational resistance to the plunger portion 100, and permit rotation of the plunger portion 100 relative to the barrel portion 200 by application of a threshold amount of force (e.g., torque) to overcome the rotational resistance. In this manner, the anti-rotational mechanism 312 does not require any additional steps by the user after rotation of the plunger portion 100 to prevent the plunger portion 100 from rotation. As described, a free state is characterized by one or more non-rack plunger facets, and therefore less force is required to axially translate the plunger portion 100 in a free state than required during a metered state in which more force is required because the one or more teeth must be overcome. Accordingly, accidental rotation from metered state to free state during injection may result in a greater amount of medicant injected than intended, increasing patient risk.

With reference to the embodiments described herein, the plunger portion 100 and anti-rotation mechanism 312 cooperate to resist plunger portion 100 rotational movement so the plunger portion 100 is not free to rotate until the holding pressure applied to the plunger portion 100 by the anti-rotation mechanism 312 to hold it in its anti-rotation orientation is overcome by application of at least a threshold amount of torque to the plunger flange 132 to purposefully twist the plunger portion 100 to change the plunger portion 100 orientation, e.g., operating mode. Embodiments include anti-rotation mechanisms 312 designed to achieve a torque threshold between about 0.1 lbf-in to about 1.0 lbf-in (in some embodiments, up to about 0.5 lbf-in), including intervening values, e.g., about 0.25 lbf-in.

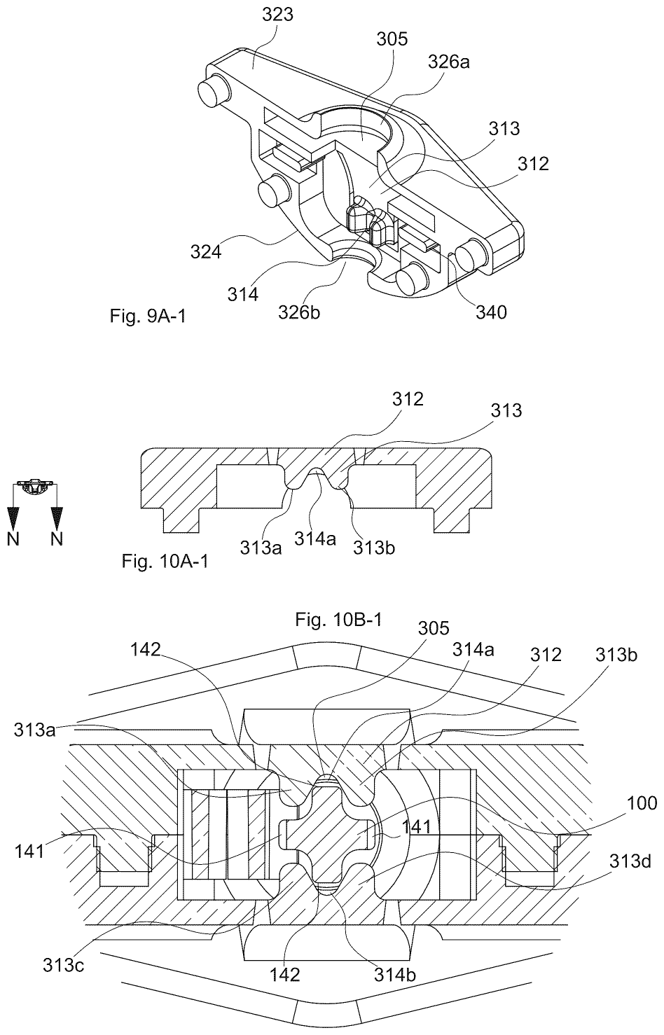

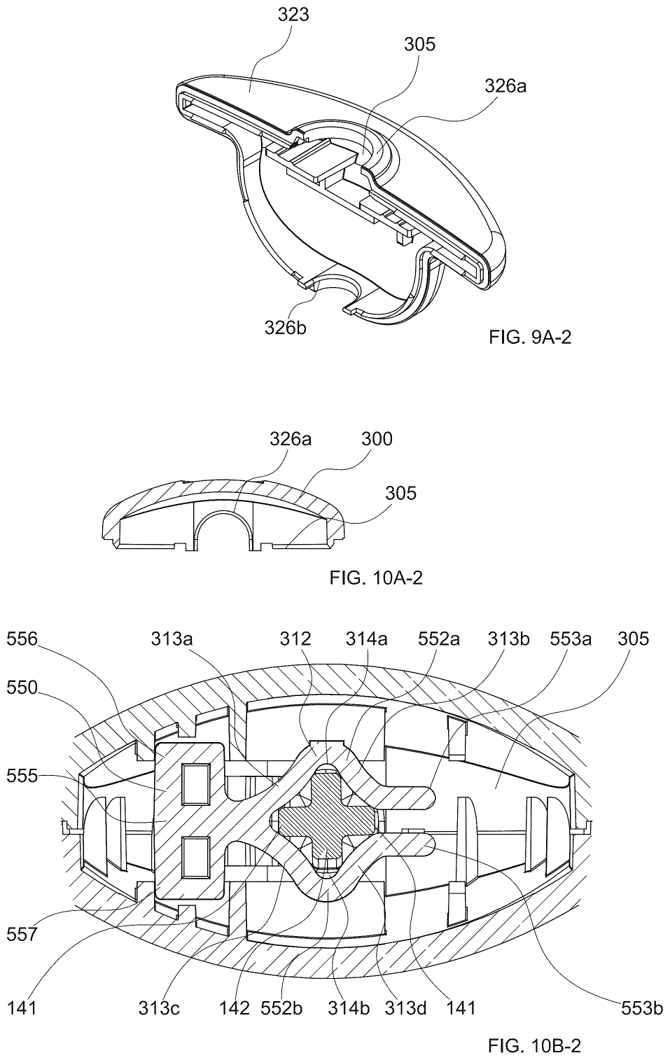

An anti-rotation mechanism 312 can be in the form of a cantilever anti-rotation mechanism 312 that extends from one or more surfaces of housing 305, e.g., from opposing surfaces (see, e.g., FIG. 9A-1, 10A-1, and 10B-1). In some embodiments, the anti-rotation mechanism is housed within grip 300, but integrated with the retention member 330, 360, 430 (see, e.g., FIGS. 9A-2 and 10A-2 illustrating the housing 305 with edges of barrel-receiving cut-away areas 326 a,b of grip, wherein the anti-rotation mechanism 312 is not illustrated as it is not formed from a surface of housing 305). Further, and as best shown for example in FIGS. 9A-1, 10A-1, 10B-1, and 10B-2, anti-rotation mechanism 312 can be in the form of one or more pair of cooperating gripping ribs 313 defined by ribs 313a, b, c, d . . . , wherein each pair of gripping ribs 313 form one or more gripping cut-outs 314a,b (channels). Specifically, FIG. 10B-1 illustrates that anti-rotation mechanism 312 can be in the form of one or more pair of cooperating gripping ribs 313 defined by ribs 313a, b, c, d, wherein the anti-rotation mechanism 312 is a separate structure from bias member 430. More specifically, FIG. 10B-2 illustrates that anti-rotation mechanism 312 can be in the form of one or more pair of cooperating gripping ribs 313 defined by ribs 313a, b, c, d, wherein anti-rotation mechanism 312 is part of the anti-rotation-bias assembly 550.

When assembled within syringe 10, the anti-rotation mechanism 312 can extend beyond the edges of barrel-receiving cut-away areas 326a,b of grip 300, as best shown in FIGS. 9A-1 and 10A-1. Those of skill in the art will recognize that greater or fewer pairs of gripping ribs 313 can be used, as well as greater or fewer ribs. Embodiments that include four-faceted plungers 100 can have two pair of complimentary gripping ribs 313a, b and 313c, d (see, e.g., FIGS. 10B-1 and 10B-2). The gripping ribs 313 cooperate to apply pressure to the plunger portion 100 so it resists rotation.

The anti-rotation mechanism 312 embodiment of FIGS. 10B-1 and 10B-2 is shown with two pair of gripping ribs 313a,b and 313c,d locking (resisting rotation of) a cruciform-shaped plunger portion 100 in its metered state with free facets 142 of the plunger 100 held in place within gripping cut-outs 314a,b. That is, a first free facet 142 of the cruciform plunger 100 is held in place within cut-out 314a by cooperating gripping ribs 313a,b of a first pair of gripping ribs, and a second free facet 142 of the plunger 100 is held in place in cut-out 314b by cooperating gripping ribs 313c,d of second pair of gripping ribs. In such a configuration, the syringe 10 is in a metered state such that the free end of the spring clip 330 or 430 is in engagement with one of the one or more rack facets 141 of the plunger portion 100 that is not within a gripping cut-out, and is locked in the metered state until at least the threshold amount of torque is applied to the plunger portion 100 to overcome the anti-rotation resistance. If rotated, e.g., 90 degrees, to a free state, a free plunger facet is not held within a gripping cavity and can be engaged with the free end of the spring clip 330 or 430, and therefore the syringe 10 is locked in the free state and prevented from rotating to a metered state. The syringe 10 therefore holds the plunger portion 100 in this selected free operating state and prevents it from rotating to a metered operating state until at least the threshold amount of torque is applied to the plunger portion 100 to overcome the anti-rotation resistance. The anti-rotation mechanism 312 can be made of an expandable material so the cut-outs can expand when torque is applied to rotate the plunger portion 100. Materials include but are not limited to polypropylene, ABS, and polycarbonate.

As described herein, and as best shown in FIGS. 1B-1 and 1B-2, plunger portion 100 is slidable between an extended position and a retracted position relative to barrel portion 200 when coupled to the barrel portion 200. Plunger portion 100 includes a first end 130 that is the proximal end, a second end 150 that is the distal end, a medial shaft 140 therebetween, and a periphery 146. Distal end 150 can include a piston 160 made of a resilient material, e.g., rubber.

Referring to FIGS. 1A to 1B-2, the piston 160 is configured to provide a slidable seal with the interior surface 202 of the medial cylinder 240 of the barrel 200 along the internal circumference of the medial cylinder 240. As the plunger portion 100 is axially translated within the barrel portion 200, the piston 160 acts as a sealant and creates a vacuum seal with the medial cylinder 240 as the plunger portion 100 is axially translated within barrel portion 200 along central axis X-X in a proximal direction (away from the distal end of the barrel 200) to withdraw medicament from a reservoir and into the syringe 10 when the syringe 10 is set to its free, or withdraw operational state, and in a distal direction (towards the distal end of the barrel 200) to eject medicament from the syringe 10 when the syringe 10 is set to its metered, or inject operational state. Embodiments include over-sized pistons 160 that are oversized relative to an inner diameter of the barrel portion 200. In such embodiments, the over-sized piston 160 is axially translated within the barrel 200 to draw liquid up or push it out, thereby causing slight expansion of the barrel 200 and creating a vacuum seal.

In some embodiments, the piston 160 pushes out all or nearly all medicament from the syringe 10, when the plunger portion 100 is at its most-distal position within the barrel 200, so that zero or near-zero volume of medicament remains—stated otherwise, zero or near-zero dead space (without an attached needle). Some syringe 10 embodiments disclosed herein include zero or near-zero syringe dead volumes that are less than about 0.02 mL, e.g., that range from about 0.009 mL to about 0.02 mL, e.g., 0.015 mL of waste, including all intervening values, without an attached needle. An example of waste measurement of an embodiment of a disclosed syringe 10 is described in Table 1.

TABLE 1

| Test # | Waste (g = mL) |
|---|---|
| 1 | 0.0093 |
| 2 | 0.0160 |
| 3 | 0.0123 |
| 4 | 0.0155 |
| 5 | 0.0158 |
| 6 | 0.0162 |
| 7 | 0.0153 |
| 8 | 0.0168 |
| 9 | 0.0143 |
| 10 | 0.0170 |
| | Average Waste = |
| | 0.015 mL |

As described, when a needle is attached, the needle hub-syringe interface can add a very small amount of dead space. Syringe 10 embodiments disclosed herein can include needle hub-syringe assemblies having zero or near-zero dead volumes (syringe 10 and needle combined) that are less than about 0.02 mL, e.g., that range from about 0.01 mL to about 0.025 mL, e.g., about 0.016 mL, including all intervening values.

An example of waste measurement of an embodiment of a disclosed needle hub-syringe assembly is described in Table 2.

TABLE 2

| Test # | Waste (g = mL) |
|---|---|
| 1 | 0.0123 |
| 2 | 0.0133 |
| 3 | 0.0164 |
| 4 | 0.0168 |
| 5 | 0.0203 |
| 6 | 0.0158 |
| 7 | 0.0161 |
| 8 | 0.0114 |
| 9 | 0.0196 |
| 10 | 0.0176 |

Average Waste = 0.016 mL

With reference to all the embodiments described herein, plunger portion 100 can be made of any suitable material and can include more than one material. For example, the plunger's distal end 150 can be made from a material that is different from one or more other material(s) of the remainder of the plunger portion 100. For example, a piston 160 can be made from a resilient material, such as, but not limited to, polymers such as rubber, polyisoprene, polyethylene, and the like, and one or more other plunger portions 100 can be made from a more rigid material such as, but not limited to, a polymer such as polyethylene, polypropylene, and polycarbonate. Plunger portion 100 and barrel portions 200 can be made of the same or different material. Embodiments include polypropylene barrels 200 and polyethylene and polycarbonate plunger portions 100 (with or without a rubber distal end). Some or all of a plunger portion 100 or a barrel portion 200 can be coated with a lubricant.

In some embodiments, the plunger 100 can be any suitable shape, including regular and irregular cross-sectional shapes, as taken perpendicular to axis X-X (FIG. 1A). Cross-sectional plunger 100 shapes include but are not limited to circular and regular and irregular polygonal shapes such as triangular, square, pentagonal, hexagonal, heptagonal, octagonal, and the like. For example, a plunger 100 can have one or more defined facets, where a facet is non-limiting and can refer to, as examples, a face, an edge (e.g., intersection of two faces) or some other geometry of the plunger 100. Facets can be equal and all of the angles may be equal or facets and/or angles can differ. Embodiments include three-faceted plungers 120 forming three faces, and four-faceted plungers 100 forming four faces, such as plungers 120 with diamond cross-sectional shapes, as taken perpendicular to axis X-X. In some embodiments, four-faceted plungers 100 can have adjacent facets disposed at right angles to one another. Facets may be curved, e.g., convex or concave, and corners and edges can be rounded.

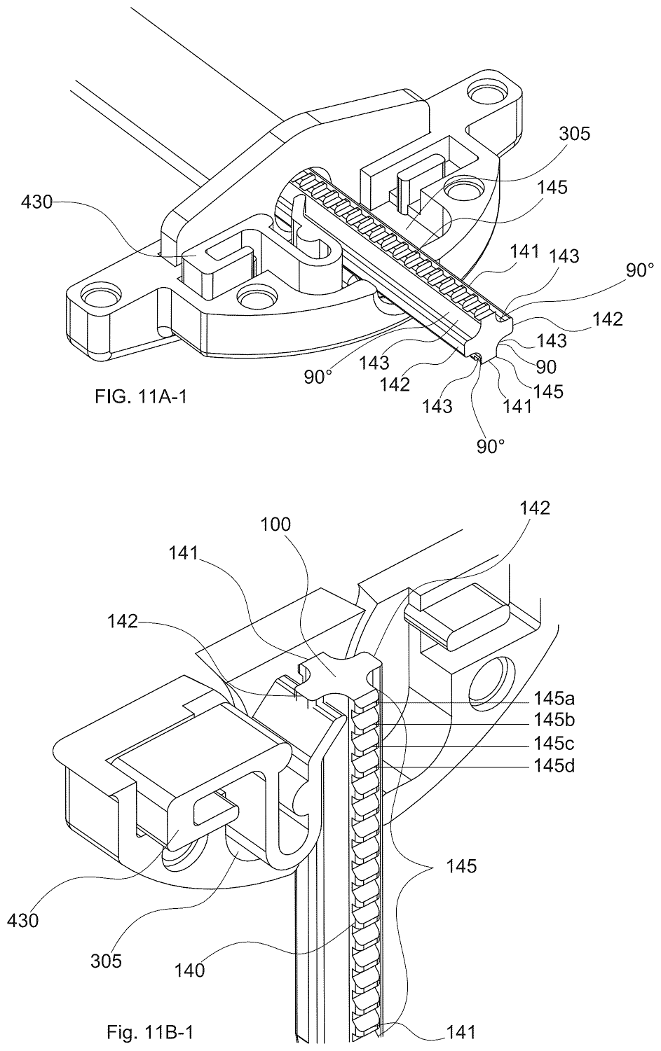

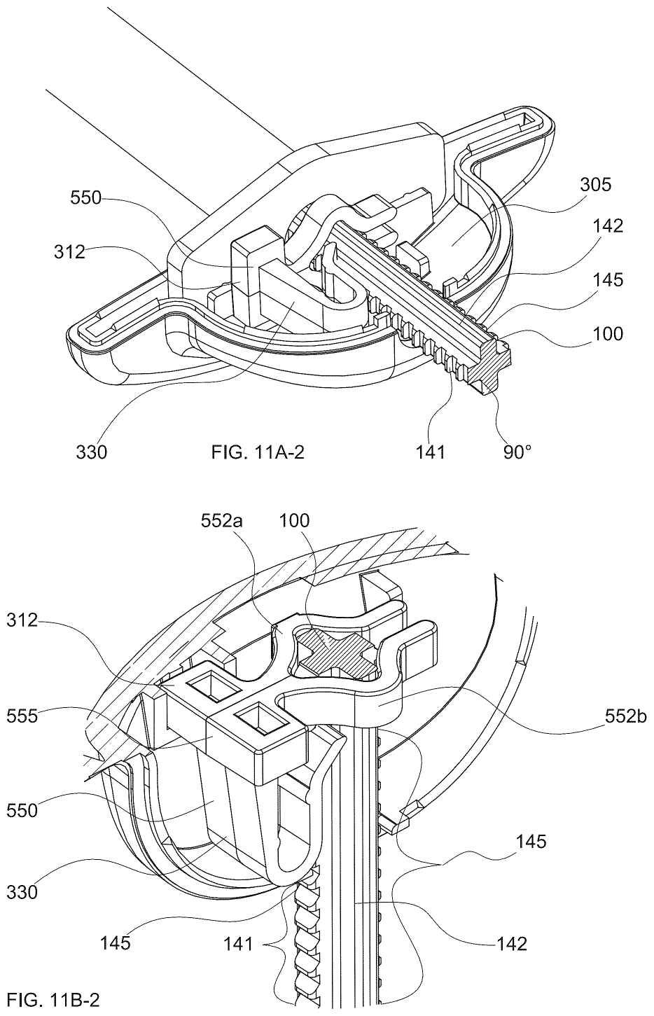

As described above, plunger medial shaft 140 includes at least one rack facet 141 (also referred to as inject facet, a ratchet-tooth rack assembly facet, or the like) that includes a plurality of metering teeth 145 and at least one free facet 142 (also referred to as non-rack facet, a withdraw facet, or the like) that does not include metering teeth (see, for example, FIGS. 1B-1, 1B-2, 2A, 2C-1, 2C-2, 3A, 3C-1, 3C-2, 4A-1, 4A-2, 5A-1, 5A-2, 8A-8D, 11A-1 to 11B-2, and 12). In some embodiments, the teeth 145 cooperate with the free end of a bias member 330 (in some embodiments, bias member 330 of anti-rotation-bias assembly 550), 360, or 430 to arrest motion of the plunger 100 at each increment. For example, free facets 142 are free of, or have an outer surface that are otherwise physically distinguishable from, the rack facets 141, in that the free facets 142 are smooth, or at least smoother, than rack facets 141. According to an aspect of the embodiments, the plunger facets interact without interruption with the free end of bias member 330, 360, or 430 during plunger portion 100 translation within the barrel portion 200. For example, in FIGS. 2C-1, 11A-1, and 11B-1, a bias member in the form of spring clip 430 is shown, wherein the plunger facets is configured to interact without interruption with the free end of spring clip 430 during plunger portion 100 translation within the barrel portion 200. Specifically, the free end of spring clip 430 can interact with teeth 145 of a rack facet 141 to provide periodic resistance during plunger portion 100 translation in a distal direction, and the spring clip free end-rack interaction can provide audible and/or tactile feedback, enabling the clinician to inject neurotoxin in desired amounts to a patient without having to visually confirm each injection by looking at the plunger 100, thereby increasing clinician focus on the patient. FIGS. 2C-2, 11A-2, and 11B-2 also illustrate an exemplar embodiment of the plunger facets interacting without interruption with the free end of bias member 330, 360, or 430 (bias member 330 of anti-rotation-bias assembly 550 shown in FIGS. 2C-2, 11A-2, and 11B-2) during plunger portion 100 translation within the barrel portion 200 in the operational state. Specifically, FIGS. 2C-2, 11A-2, and 11B-2 illustrate the free end of bias member 330 interacting with the plunger facets (rack facets 141), wherein in this exemplar embodiment, the bias member 330 is integrated with the anti-rotation mechanism 312.

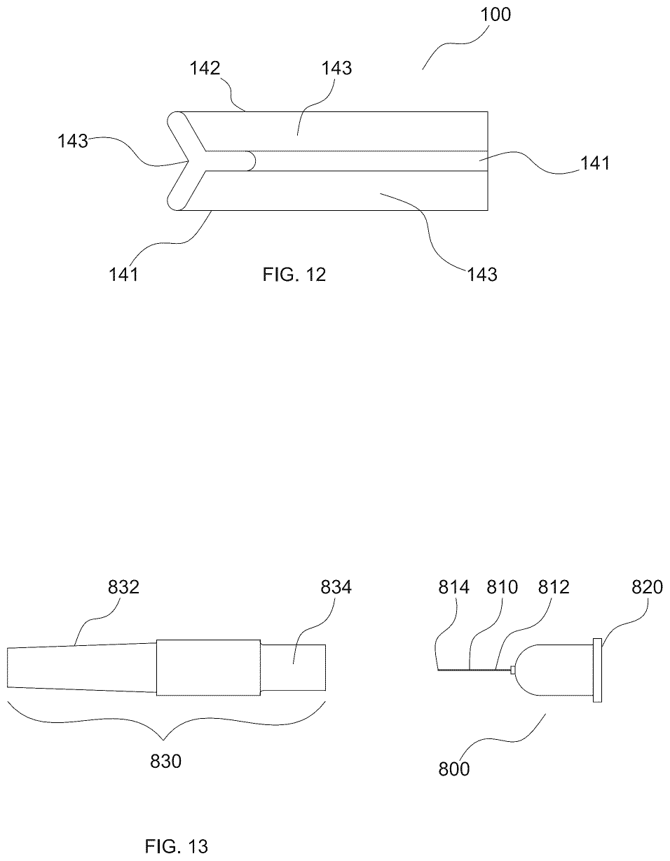

In some embodiments, a plunger 100 can have three distinct facets, wherein each of the three facets can be selectively positioned to interact with the free end of the spring clip 330 (in some embodiments, bias member 330 of anti-rotation-bias assembly 550) or 430 when a respective one of the facets is positioned in relation to the spring clip 330 or 430 free end. A three-faceted plunger 100 can have a trilobal cross-sectional shape, as shown in the embodiment of FIG. 12. The trilobal embodiment of FIG. 12 can have two rack facets 141 defining two metered operational states, and one free facet 142 defining a free operational state. Embodiments also include trilobal plungers 100 having two free facets 142 and one rack facet 141. The embodiment of FIG. 12 shows racked (metered) facets 141 and free facets 142 positioned at the edges of the trilobal plunger 100, however, one or more can be positioned at one or more of the hollowed-out areas 143 instead of or in addition to at the edges.

According to some embodiments, a plunger 100 can have four distinct facets and each can be selectively positioned to interact with the free end of the spring clip 330 or 430 when a respective one of the facets is positioned in relation to the free end. Two facets of a four-faceted plunger 100 can define free facet 142 defining two free operational states, and the two other facets can define rack facets 141 defining two metered operational states. In other embodiments of a four facet plunger 100, one facet can define a free operational state and three facets can define three metered operational states, or three facets can define three free operational state and one facet can define a metered operational state.

In some embodiments, the four-faceted plunger 100 can be in the shape of a cruciform having a cruciform cross-sectional shape, as taken perpendicular to axis X-X, in which each facet of the cruciform plunger 100 can be equal and each angle may be equal, and each facet of the cruciform plunger 100 can be at 90 degrees to the other. Accordingly, four-faceted embodiments such as cruciform-shaped plungers 100, can comprise the rack facets 141 opposite each other, and the free facets 142 opposite each other. In this manner, the syringe 10 is configured so that twisting the plunger portion 100 90-degrees in either direction will result in a metered state. An embodiment of a cruciform plunger 100 is shown, e.g., in FIGS. 1B-1, 1B-2, 8A-8B, 10B-1, 10B-2, and 11A-1 to 11B-2.

According to some embodiments, and as best depicted in FIGS. 11A-1 to 11B-2, the cruciform plunger 100 can include two rack facets 141 such that each rack facet 141 has a rack 145 (also referred to as a plurality of metering teeth 145, and the like) with metering teeth 145a, 145b, 145c, 145d . . . , two free facets 142 such that each free facet 142 is free of a rack (free of the metering teeth 145), and four hollowed-out areas 143. The cruciform plunger's rack facets 141 can be adjacent each other or may be opposite each other, e.g., separated by free facets 142. In like manner, the free facets 142 can be adjacent each other or can be opposite each other, e.g., separated by rack facets 141. A first rack facet 141 can be at 90 degrees to a first free facet 142, which can be at 90 degrees to a second rack facet 141, which can be at 90 degrees to a second free facet 142. In other words, the two rack facets 141 can form a pair of facets that are each perpendicular to each of the pair of free facets 142. A cruciform plunger 100 facilitates anti-rotation wherein one or more of the plunger facets engage with gripping ribs 313 to prevent rotation.

As shown in the embodiment of FIG. 7F, a four-faceted plunger 100 can also be in the shape of a diamond having a diamond cross-sectional shape, as taken perpendicular to axis X-X, in which each facet of the diamond plunger 100 can be equal and each angle can be equal, and each facet of the square or diamond plunger 100 can be at 90 degrees to the other. A diamond-shaped plunger lacks hollowed-out areas 143 (not shown) of a plunger 100 having a cruciform cross-sectional shape.

Still referring to FIG. 7F, a diamond-shaped plunger 100 can include two rack facets 141 such that each rack facet 141 has a rack 145, and also include two free facets 142 such that each free facet 142 is free of a rack 145. In some embodiments, a diamond plunger's 100 rack facets 141 can be adjacent each other or may be opposite each other, e.g., separated by free facets 142, and likewise the free facets 142 can be adjacent each other or may be opposite each other, e.g., separated by rack facets 141. In some embodiments, a first rack facet 141 can be postioned at 90 degrees relative to a first free facet 142. Further, the first free facet 142 can be at a 90-degree angle relative to a second rack facet 141, wherein the second rack facet 141 can be at a 90-degree angle relative to a second free facet 142. As such, the two rack facets 141 can form a pair of facets that are each perpendicular to each of the pair of free facets 142. A diamond plunger 100 can also facilitate interaction with the anti-rotation mechanism wherein one or more of the plunger facets engage with gripping ribs 313 to prevent rotation.

The thin facets of a plunger 100, e.g., of a cruciform cross-section shaped plunger 100, trilobal cross-section shaped plunger 100, and a diamond cross-section shaped plunger 100, provide a reduced contact area between the plunger 100 and the free end of the spring clip 330 or 430. The reduced contact area minimizes friction between the plunger 100 and the free end of the spring clip 330 or 430 allowing for smoother axial translation, e.g., when in the free state. As described, the edges of a plunger 100 with a diamond cross-section (where two broader facets adjoin) can serve as rack and non-rack facets to define the operational states of the plunger portion 100 and to provide a reduced plunger-spring clip contact area relative to using a broader plunger facet (see e.g., FIG. 7F). The broader facets of a cruciform plunger portion 100 and edges of a diamond plunger 100 can be rounded to further reduce contact or improve smooth operation.

As best shown, e.g., in FIGS. 4A-1, 4A-2, 5A-1 to 5K, 8A, 8B, and 11A-1 to 11B-2, plurality of teeth 145 includes a series of teeth 145 spaced apart from each other and positioned along a plunger facet 141 (e.g., each tooth shown as 145a,b, c, d in FIG. 5A-1 and 5A-2) evenly spaced apart along one or more facets 141 of a plunger medial shaft 140. In some embodiments, the spacing between teeth 145 is calibrated to incrementally dispense precise volumes of medicament upon axial translation of the plunger 100 in a distal direction to cause advancement of the free end of the spring clip 330, 430 to the next tooth 145 of the plurality.

The spacing of the teeth 145 is designed so that the user does not accidentally skip increments, even when the increments are extremely closely spaced together, e.g., a small volume syringe having small spacing between teeth 145. Dimensions can be the same or different on different facets 141 of a plunger 100.

As best shown in the exemplar embodiment depicted in FIGS. 5A-1 and 5A-2, each tooth 145a,b, c, . . . of a plurality includes distal-facing surface 155a (also referred to as the back side) and proximal-facing inclined surface 155b (also referred to as the front side). Spaces formed between successive teeth 145 define rack gaps G (each gap shown as gap G1, G2, G3 . . . ) along at least a portion of a metered plunger facet. The teeth 145 of a rack facet of the plunger 100 consistently and progressively engage with the end of the spring clip 430 during axial translation in periodic fashion when in a metered state. Likewise, at least one facet of the plunger 100 is a free facet 142 without teeth 145 and is configured to consistently contact the end of the spring clip 430 during axial translation when in a free state. Accordingly, regardless of the state of the plunger 100, the free end of the spring clip 330 or 430 is always in uninterrupted contact with a facet of the plunger 100 (see, e.g., FIGS. 6C, 7A, 8C, 8D, and 11A-1 to 11B-2). The single action of axially advancing the plunger 100 in the distal direction by application of a pushing force to the plunger flange 132 causes the spring clip 330 or 430 to travel out of a rack gap G and over the surfaces of a tooth 145 to a more-proximal tooth 145. In this manner, the spring clip 330 or 430 free end remains in constant, uninterrupted contact with the plunger 100 during axial translation. The uninterrupted contact of the free end with the plunger 100 obviates the need to engage and then entirely disengage the spring clip 330 or 430 from the plunger 100 to advance the plunger 100 for successive injections, and obviates the need to increment the injections using a separate switch and/or grip 300. A separate switch/grip 300 adds to cost, complexity, and distraction, particularly if manual manipulation of the separate switch/grip 300 is required.

Embodiments include teeth 145 spacings F of about 0.02 inches to about 0.1 inches (e.g., 0.045 inches), tooth 145 angles H of about 120 degrees to about 150 degrees, tooth 145 backside angles I of about 90 degrees to about 120 degrees, and tooth 145 depths J of about 0.01 inches to about 0.05 inches. These dimensions are useful for some 1 mL syringe 10 embodiments, e.g., and enables the spring clip 330 or 430 to release from a tooth 145 and travel immediately to impact the next tooth 145.

For example, some embodiments of a 1 mL volume syringe 10 configured to increment medicament in 0.02 mL increments can have an inner diameter of about 0.19 inches, tooth 145 spacings F of about 0.045 inches apart, tooth 145 angles H of about 135 degrees, tooth 145 backside angles I of about 90 degrees, and tooth 145 depths of about 0.02 inches. In such embodiments, teeth 145 uniformly spaced apart on a rack facet 141 can correspond to a syringe 10 that injects medicament in increments of 0.02 mL of liquid injected for each advancement of the spring clip 330 or 430 to an adjacent tooth 145 of the rack 145, for example for a barrel 200 having an inner diameter of about 0.19 inches. In other words, one audible snap caused by the interaction of the spring clip 330 or 430 as it overcomes a single tooth 145 can correspond to 0.02 mL of medicament injected from the syringe 10.

Embodiments described herein include syringes 10 configured to dispense amounts other than 0.02 mL incremented injections, e.g., 0.01 mL, 0.02 mL, 0.025 mL, 0.04 mL which are useful for botulinum toxin injections, for example, although it is to be understood that the disclosure includes other syringes 10 configured to increment other volumes. As described, different rack facets 141 of a plunger 100 can have the same spacing of the teeth 145 of the rack or may have different spacing, and therefore different rack facets 141 can increment the same or different amounts of medicament.

As a non-limiting example, a 1 mL volume syringe 10 configured to increment medicament in 0.025 mL increments can have an inner diameter of about 0.19 inches, tooth 145 spacings F of about 0.054 inches apart, tooth 145 angles H of about 135 degrees, tooth 145 backside angles I of about 90 degrees, and tooth 145 depths of about 0.02 inches. In such embodiments, teeth 145 uniformly spaced apart on a rack facet 141 can correspond to a syringe 10 that injects medicament in increments of 0.025 mL of liquid injected for each advancement of the spring clip 330 or 430 to an adjacent tooth 145 of the rack, for example for a barrel 200 having an inner diameter of about 0.19 inches. In other words, one audible snap caused by the interaction of the spring clip 330 or 430 as it overcomes a single tooth 145 can correspond to 0.025 mL of medicament injected from the syringe 10.

The progression of the spring clip 430 travelling over teeth 145 of a rack is shown in FIGS. 5B-5F. FIG. 5B shows the free end of the spring clip 430 at rest in a gap G formed by the spacing between teeth 145. FIG. 5C shows the free end of spring clip 430 sliding up the front facet of a tooth 145. FIG. 5C shows the free end of the spring clip 430 at the apex of the tooth 145. FIG. 5D shows the free end of the spring clip 430 released from the tooth 145 and traveling to impact the next tooth 145. FIG. 5D shows the free end of the spring clip 430 impacting with the next tooth 145 to cause an audible sound and tactile feedback.

Further, the progression of the bias member 330 of an anti-rotation-bias assembly 550 travelling over teeth 145 of a rack is shown in FIGS. 5G-5K. FIG. 5G shows the free end of the bias member 330 at rest in a gap G formed by the spacing between teeth 145. FIG. 5H shows the free end of bias member 330 sliding up the front facet of a tooth 145. FIG. SI shows the free end of the bias member 330 at the apex of the tooth 145. FIG. 5J shows the free end of the bias member 330 released from the tooth 145 and traveling to impact the next tooth 145. FIG. 5K shows the free end of the bias member 330 0 impacting with the next tooth 145 to cause an audible sound and tactile feedback.

In some embodiments, the plunger's proximal end 130 includes proximal plunger flange 132 (best shown in FIGS. 1A to 3C-2). Actuation of flange 132 causes longitudinal and rotational movement of plunger 100 relative to barrel 200, and therefore a clinician can control plunger 100 axial translation and operational mode by interaction with flange 132. Flange 132 has proximal flange surface 134 that can be used as a location for a clinician's finger/thumb, e.g., to push the plunger 100 in a distal direction, and an opposite distal flange surface 135 which can be used to help pull the plunger 100 in a proximal direction.

According to some aspects of the embodiments, flange 132 (best shown in FIG. 1A to 3C-2) can be an indicator of the operational state of the syringe 10. Flange 132 can be sized and/or shaped and/or positioned relative to the barrel 200 to indicate a plunger's 100 operational state. A flange 132 can include one or more of a visual, tactile and audible indicator of plunger 100 operational mode, including but not limited to arrows, lines, symbols, text, and the like. The position of flange 132 relative to grip 300 (and/or other part of the syringe) can be an indicator. An indicator can be one or more distinguishable flange 132 portions that correspond with a specific facet of the plunger 100 to provide quick and easy visual confirmation of the selected operational state of the plunger 100.

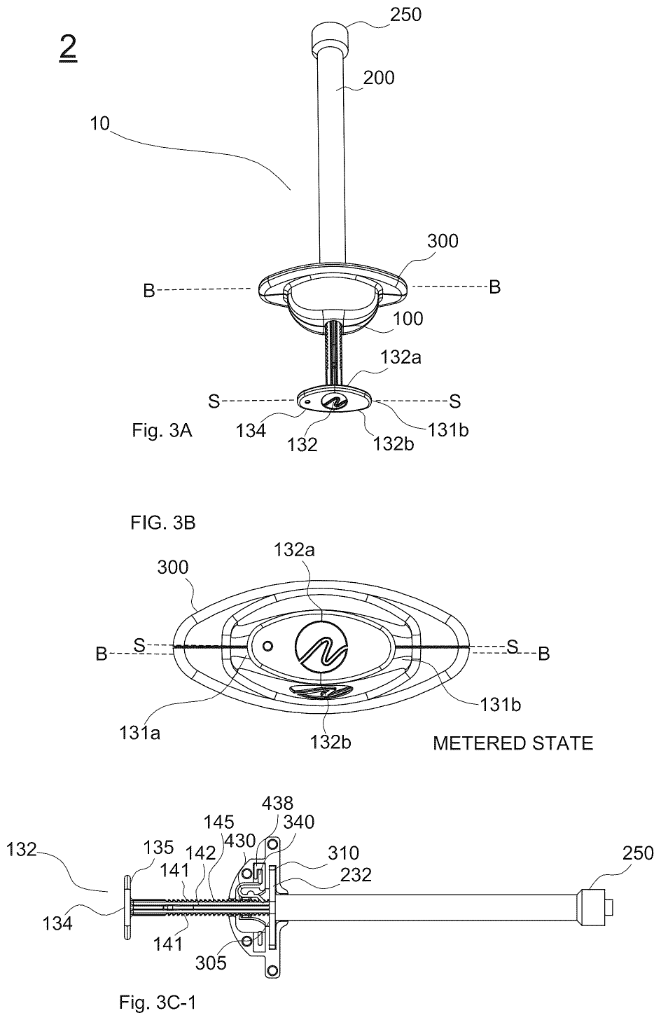

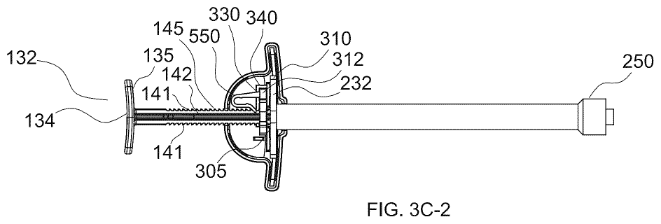

Embodiments include asymmetrical flanges 132, and the mode of operation of a syringe 10 can be easily visually and/or tactilely identified via the flange 132 asymmetry. For example, a flange 132 can define a long axis S-S (see, e.g., FIGS. 2A, 2B, 3A, 3B and 16). A flange 132 can have the same number of sides as the plunger 100 has facets 141, 142. For example, a four-sided flange 132 of a four-faceted cruciform plunger 100 can have two metered flange sides 131a,b that are each fixedly positioned to line-up (register) with each of two plunger rack facets 141, and two free flange sides 132a,b that are each fixedly positioned to line-up (register) with each of two plunger free facets 142. The flange 132 can be asymmetrical and metered flange sides 131a,b of the four-sided flange can be minor sides and the free sides 132a,b can be major sides, or vice versa, or the sides may be otherwise distinguishable (i.e., in other words, they can be asymmetrical). Various positions of the flange 132 relative to grip 300 can therefore provide a visual cue of the selected operational state of the plunger 100. For example, as illustrated by FIGS. 2A, 2B, 3A, 3B, and 16, a flange 132 having long dimension axis S-S positioned perpendicular to grip 300 long dimension axis B-B can indicate that the free end of the spring clip 430 is in contact with a free facet 142 of the plunger 100 and that the syringe 10 is therefore in the free state (FIGS. 2A and 2B) (for example, to withdraw liquid into the syringe 10). In like manner, a flange 132 long dimension axis S-S positioned parallel to grip 300 long axis B-B can indicate that the free end of the spring clip 430 is in contact with a rack facet 141 of the plunger 100 and the syringe 10 is therefore in the metered operational state (FIGS. 3A to 3C-1), for example to inject liquid into a patient, or vice versa.

Specifically, in some embodiments, as best shown in FIG. 3C-1, the free end of bias member 330, 360, or 430 (bias member 430 illustrated in FIG. 3C-1) is in contact with a rack facet 141 of the plunger 100 and the syringe 10 when in the metered state. FIG. 3C-2 illustrates an additional exemplar embodiment, wherein the free end of bias member 330, 360, or 430 (bias member 330 illustrated in FIG. 3C-2) is in contact with a rack facet 141 of the plunger and the syringe 10 when in the metered state, and wherein the fixed end of bias member 330 is integrated with the anti-rotation mechanism 312 and in contact with the retention feature 340.

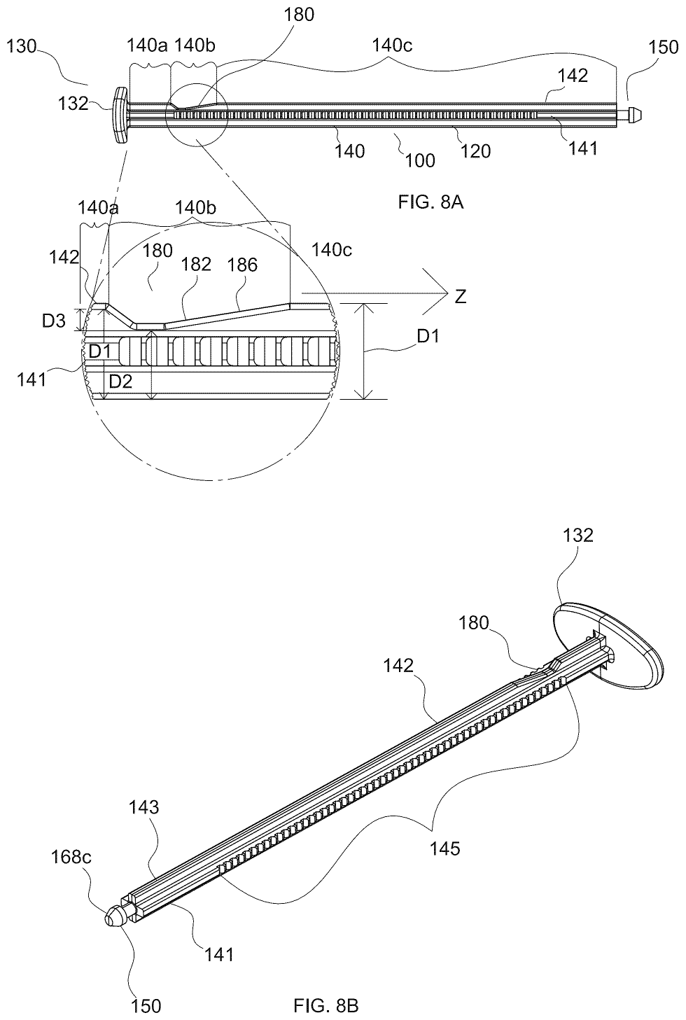

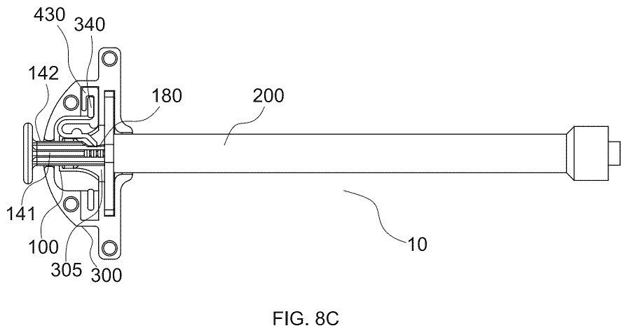

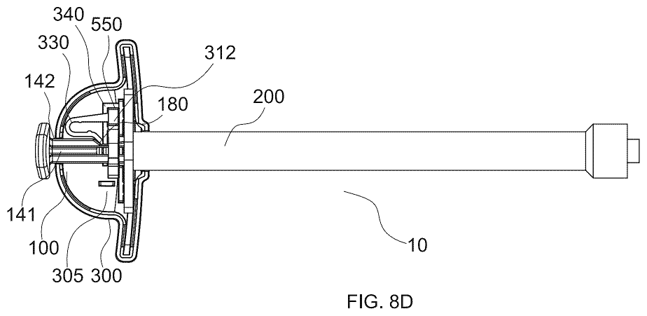

In some embodiments, and as best shown in FIGS. 8A-8D, plunger medial shaft 140 can include a spring clip relief 180 configured to receive the free end of a spring clip 330, 430 (best shown in FIGS. 8C and 8D) and maintain the clip 330 (FIG. 8D) or 430 (FIG. 8C) in an un-stressed or low stress state ((for example, during sterilization and storage). This is particularly useful for polymer spring clips 330 or 430 and increases the reliability of the spring clip 330 or 430. The relief 180 is configured to avoid engagement of the spring clip 330, 430 with a non-relief portion of the plunger shaft 140 when the plunger 100 is fully advanced within the barrel 200.

In some embodiments, and with reference to FIGS. 8A-8D, the relief 180 can be located on a section of one of the plunger facets that is free of teeth 145. This can be on a section of at least one free facet 141 of the plunger 100, or a section of at least one rack facet 142 that does not include teeth 145 such as a non-tooth section proximal of a plurality of teeth 145. Embodiments include spring clip reliefs 180 positioned at the proximal end 130 of medial shaft 140, including reliefs 180 positioned at the proximal end 130 of medial shaft 140 on at least one free facet 142 of the plunger 100, so that a spring clip 330, 430 can engage with the relief 180 and is maintained in an un-stressed state when engaged. For example, a spring clip 330, 430 can rest in a relief 180 positioned on a free facet 142 of the plunger 100 and assume an unstressed or low stressed state when plunger portion 100 is fully advanced within barrel 200 and until the spring clip 330, 430 is moved from the relief 180 and stressed. As such, a low stressed state can be relative to a stressed state that is assumed when the spring clip 330 or 430 is moved from the relief 180 and put under stress. In some exemplar embodiments, medial shaft 140 can include first section 140a between plunger flange 132 and relief 180 (proximal of relief 180), second section 140b that includes relief 180, and third section 140c that is distal of relief 180, e.g., between relief 180 and piston 160 (best shown in FIG. 8A), wherein relief 180 is positioned on free facet 142.

FIGS. 8A-8D show relief 180 in the form of a necked-down region (notch) of the plunger 100 having a reduced outer diameter, as compared to the outer diameter of the plunger 100 more-distal and/or more proximal to the relief 180. The outer diameter of sections 140a,c are necked-down to form the relief 180. In some embodiments, relief 180 can have an upwardly-sloping ramp that gradually transitions from relief 180 diameter D2 to plunger 100 outer diameter D1 and to section 140c of the plunger 100. For example, FIG. 8C shows the syringe 10 with one half of grip 300 removed and the free end of the spring clip 430 unbiasedly resting in relief 180 in a relief state, e.g., a sterilization and/or shelf state. In like manner, FIG. 8D shows the syringe 10 with one half grip 300 removed and the free end of the spring clip 330 of the anti-rotation-bias assembly 550 unbiasedly resting in relief 180 in a relief state, e.g., a sterilization and/or shelf state. With particular reference to FIGS. 8A-8D, the reduced outer diameter D2 of the relief 180 unbiases the spring clip 330, 430 (e.g., it does not bias the spring clip 330, 430). The spring clip 330, 430 is moved out of the relief 180 by at least one of axial translation and rotational movement. For example, axial translation of the plunger 100 relative to the barrel 200 in the proximal direction, causes the free end of the spring clip 330, 430 to move out of the relief 180 by traveling over relief surface 182 and over ramp 186 to transition to section 140c, shown by arrow Z (FIG. 8A). The transition gradually stresses the spring clip 330, 430 as the outer diameter of the plunger 100 increases and biases the spring clip 330, 430. Relief 180 depth D3 (FIG. 8A) can be greater than the depth of the teeth 145.

Prior to clinical use, the free end of the spring clip 330 or 430 can rest completely in the relief 180 and when liquid is drawn up into the syringe 10 by pulling back the plunger portion 100, the spring clip 330 or 430 is loaded under force and deflected by an amount the same or greater than the depth of the reliefs 180. Accordingly, even when the spring clip 330 or 430 is moved from the relief 180 and positioned to rest in a rack gap G, it is still loaded under force.

In the embodiments described herein, a syringe 10 can be in a relief state before or during sterilization and/or post-sterilization shelf storage, e.g., placed in the relief state by a manufacturer and provided to a clinician in this relief state. This eliminates stress on the spring clip 330 or 430 during one or more pre-use stages and prolongs its optimal effectiveness when it is used. A syringe 10 can have a plastic spring clip 330 or 430 and a plunger portion 100 with corresponding plunger relief 180. In some embodiments, the plastic spring clip 330 or 430 can be positioned to rest in a plunger relief 180 before clinician use, such as before and during shelf life storage, i.e., before clinical use. When retrieved from storage for clinical use, the plunger portion 100 is advanced to move the free end of the spring clip 330 or 430 out of the relief 180, at which point the spring clip 330 or 430 undergoes constant, uninterrupted contact with a non-relief section of at least one free facet 142 and/or at least one rack facet 141 of the plunger 100. The spring clip 330 or 430 maintains constant contact with the plunger 100 even when in a relief state. The relief 180 can be designed such that a small amount of interference exists between plunger 100 relief area and spring clip 330 or 430, while effectively reducing stress on the spring clip 330 or 430 during one or more pre-use stages.

As described, embodiments include near-zero waste syringes 10. In contrast, conventional syringes can accumulate significant amount of waste within the in area of the distal end of the syringe that cannot be pushed out, and between the end of a needle hub and the distal-most surface of a conventional syringe. Conventional syringe waste can amount to as much as about 0.04-0.05 mL, e.g., up to about two units of medicament can be lost to waste. This waste is significant for expensive injections such as botulinum toxin, and conventional fixed-needle syringes don't address all of the waste issues. However, syringes 10 disclosed herein have zero or near-zero dead space plunger 100 tips. In some embodiments, plunger distal end 150 has a distal-most tip configured to closely fit within an interior surface of the barrel 200 neck, as shown e.g., in FIGS. 1B-1, 1B-2, 4A-1, 4A-2, and 8B. As described herein, this enables nearly all medicament to be ejected from the syringe 10.

In some exemplar embodiments, and as best depicted in FIG. 1B-1 and 1B-2, plunger distal end 150 includes distal end piston 160. Piston 160 includes piston seal 165 having periphery 168a, and piston lead 168 having periphery 168b. Some embodiments include two-piece pistons 160, and therefore can include plunger tip connector 168c. Piston 160 forms a sealing arrangement with the circumference of barrel inner surface 202, including with reduced diameter neck 260 of barrel portion 200, to form a zero or near-zero dead space syringe 10. Accordingly, piston lead 168 occupies the full space of the barrel's piston space 262 when the plunger 100 is fully advanced within the barrel 200. Periphery 168a of seal 168 forms a liquid-tight, sliding vacuum seal with the circumference of barrel inner surface 202, and periphery 168b of piston lead 168 forms a liquid-tight, sliding vacuum seal with the circumference of inner surface 202a of the reduced diameter neck 260 of the barrel 200 when it is seated within piston space 262 (when the plunger 100 is fully advanced within the barrel 200) to occupy piston space 262. The occupation of space 262 by lead 168 of piston 160 when the plunger 100 is fully advanced within the barrel 200 ensures zero or near-zero dead volume and configures the syringe 10 to dispense all or nearly all of the medicament from the syringe 10 when the plunger 100 is fully advanced within the barrel 200.

FIG. 13 shows an example needle element 800 for use with the disclosed syringes 10 (not shown in FIG. 13) to provide near-zero dead space syringe-needle assemblies. Needle element 800 comprises a needle 810 and needle hub with luer-lock interface 820. In some embodiments, a needle protector 830 covers the needle element 800 before injections to avoid accidental needle stick and maintain sterility. Needle protector 830 includes needle shield 832 and needle hub cover 834. Needle 810 includes shaft 812 and tip 814, and can include a lumen. In some exemplar embodiments, tip 814 can be blunt or beveled. For injection of a neurotoxin, such as botulinum toxin into a patient, the needle can be a fine gauge needle equal to or finer than 30 gauge (“G”) (e.g., 30 G, 31 G, 32 G, 33 G, etc.), for example a 33 G×⅜ inch hypodermic needle. Luer-lock needle assembly 800 connects to luer-lock interface 290 of a syringe 10 (not shown in FIG. 13) to provide a near-zero dead space syringe-needle assembly.

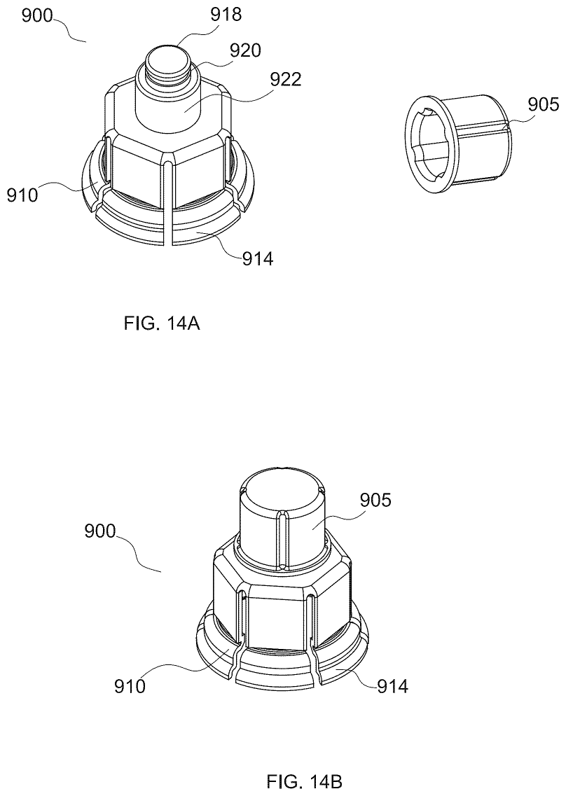

FIGS. 14A-14B show an example embodiment of a medicament vial adapter 900 for use with the disclosed syringes 10 (not shown in FIGS. 14A-14B) to provide near-zero dead space syringe-adapter assemblies. Vial adapter 900 is a needle-free adapter, in that it is configured to connect to a syringe 10 and transfer liquid such as neurotoxin from a medicament vial to the syringe 10 without the use of a needle. Vial adapter 900 can be used with any size medicament vial, e.g., 20 mm and 13 mm vials. Adapter 900 includes optional cap 905, vial attachment body 910 comprising a vial-connecting end 914 that can be configured with a snap-on connection for fastening to a medicament vial, and syringe-connecting end 918 with luer-lock interface 920. In some embodiments, luer-lock interface 920 connects to luer-lock interface 290 of a syringe 10 to provide a near-zero dead space syringe-vial adapter assembly. Luer-lock interface 920 can include a valve, which can be a swabable valve, to maintain the seal on the adapter 900 and sterility of the contents of a connected medicament vial in procedures requiring the vial to be accessed multiple times. In some embodiments, a swabbable adapter valve surface may not be necessary for embodiments that include a cap 905. The syringe-connecting end 918 can include low priming volume area 922. In some exemplar embodiments, vial adapter 900 can be 20 mm or 30 mm. The syringe-vial adapter assembly can have a near-zero dead volume (syringe 10 and adapter 900 combined) of about 0.1 mL or less, e.g., about 0.06 mL or less.

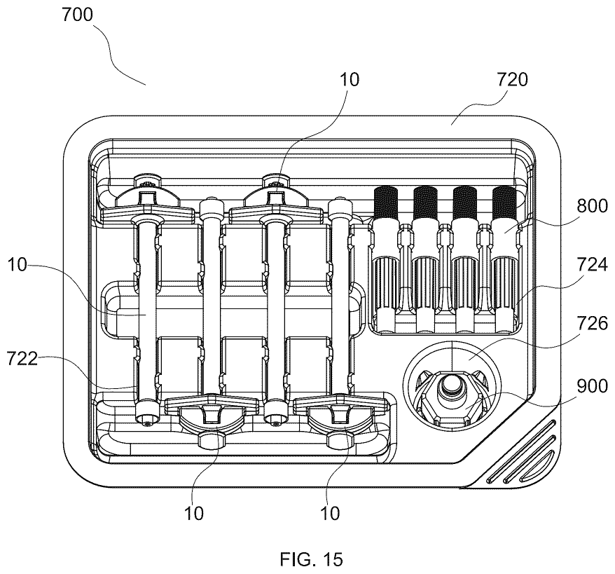

Embodiments include various injection components described herein, including kits for single or multiple botulinum toxin procedures. An example kit for single use, one patient for a botulinum toxin procedure, e.g., for cosmetic procedure, is shown in FIG. 15. Kit 700 includes components for botulinum injections, including preparation and delivery. In some exemplar embodiments, kit 700 include one or more syringes 10, one or more needle elements 800 each comprising a needle protector 830 (not depicted in FIG. 15), and one or more vial adapters 900. The embodiment of FIG. 15 shows four 1 mL syringes 10, four near-zero dead space volume needle elements 800 each having a 33G×⅜ inch hypodermic needle 810, and one vial adapter 900. In some embodiments, kit 700 can also include a vial of botulinum toxin, e.g., a 20 mm or 13 mm vial of botulinum toxin and/or can include a diluent syringe 10 prefilled with the saline required for a procedure. A pre-filled diluent syringe 10 obviates the need to draw up diluent from a saline vial and therefore reduces procedural steps. The components can be positioned in a tray 720, with each component in a defined pocket of the tray 720, pocket(s) 722 for one or more syringes 10, pocket(s) 724 for one or more needle assemblies 800, and pocket(s) 726 for one or more vial adapters 900. In some embodiments, the tray 720 can include a cover (not shown). A kit 700 may further include at least all syringe 10, needle assembly 800 and vial adapter 900 components necessary for administration of one vial of botulinum toxin.

Embodiments include methods of administering neurotoxin, e.g., botulinum toxin, to a patient by a user, wherein the methods include connecting a vial adapter 900 to a vial containing neurotoxin solution, connecting via a luer-lock interface 920, a syringe 10 without a needle to the vial adapter 900, drawing an amount of neurotoxin solution into the syringe 10, disconnecting the syringe 10 from the vial adapter 900, connecting, via a luer-lock interface 290, a needle to the syringe 10. Methods may include dispensing all or nearly all of the withdrawn amount of neurotoxin from the syringe 10 to the patient. Methods may include dispensing all or nearly all but about 0.001 to about 0.02 mL, including intervening values, e.g., 0.015 mL, mL of the withdrawn amount of neurotoxin from the syringe 10 to the patient. Methods may include loading a bias member 330, 430, 360 under force by movement of the plunger portion 100. Methods can include resting a bias member 330, 430, 360 in an unbiased or low biased state in a plunger relief 180, and loading a bias member 330, 430, 360 under force by movement of the plunger 100 out of the relief 180. Methods may include no periodic resistance experienced by the user during drawing of the amount of neurotoxin solution into the syringe 10. Methods may include providing periodic resistance during dispensing of the neurotoxin solution to the patient, e.g., and may include periodic resistance experienced by the user during dispensing of the neurotoxin solution to the patient. Methods may include preventing a change in operational state of the syringe 10 until a threshold amount of torque is applied to the syringe 10, e.g., a plunger 100 of the syringe 10, and applying a threshold amount of torque to the syringe 10, e.g., the plunger 100, to rotate the plunger 100 to change its orientation relative to the syringe barrel 200, e.g., to change the operational state of the syringe 10. Methods may include rotation of a plunger 100 of the syringe 10 to switch from a state of no periodic resistance to a state of periodic resistance. Methods may include an about 90-degree rotation of the plunger 100. Methods may include the periodic resistance enabling the user to dispense the neurotoxin solution in desired amounts to the patient without visual confirmation. Methods may include the periodic resistance enabling the user to dispense the neurotoxin solution in desired amounts selected between increments of about 0.01 mL and about 0.04 mL, including intervening values, to the patient without visual confirmation. Methods may include dispensing desired amounts in increments of about 0.02 mL. Methods may include desired amounts in increments of about 0.025 mL. Methods may include connecting, via a luer-lock interface 820, a 30 gauge or smaller needle 810, and the needle 810 is not substantially dulled from passing through a vial septum. Methods may include setting the syringe 10 to a free (withdraw) state and/or confirming it is in a withdraw state, e.g., by confirming the plunger 100 indicates a free state such as by visually observing its asymmetry relative to the grip 300. Methods may include, if not yet fully inserted, fully inserting the plunger portion 100 of a syringe 10 in the barrel portion 200 of the syringe 10, and before or after the insertion, positioning the syringe 10 in a free operational state such that a free facet 142 of the plunger 100 is engaged with a free end of a bias member 330, 430, 360 of the syringe 10. Methods may include using a spring clip bias member 330 or 430 and moving a spring clip 330 or 430 away from a spring clip relief 180 of the syringe 10 to another non-relief portion of the plunger 100 to load it under force. Methods may include moving a spring clip 330 or 430 from a spring clip relief 180 to a rachet-tooth rack assembly of the syringe 10. Methods may include, with the syringe 10 in the free state and the plunger 100 fully inserted into the syringe barrel 200, loading the syringe 10 with neurotoxin by connecting the distal end of the syringe 10 without a needle to a vial adapter 900 connected to a vial of neurotoxin and drawing the plunger 100 back (proximal direction) to load the syringe 10 with the desired amount of neurotoxin from the vial-adapter assembly. Methods may include rotating the plunger 100 to at least one facet of a plunger 100 of the syringe 10 that is a rack facet 141 to set the syringe 10 in a metered operational state, e.g., 90 degrees, to position the free end of the spring clip 330 or 430 in contact with the rack facet 141 of the plunger portion 100. Methods may include holding the plunger 100 in an anti-rotational state and applying at least a threshold amount of torque to the plunger 100 to overcome the anti-rotational resistance, and methods may include applying at least a threshold amount of torque of between about 0.1 lbf-in to about 1.0 lbf-in (in some embodiments, up to about 0.5 lbf-in), including intervening values, applied to the plunger 100 to overcome the anti-rotational resistance. Methods may include positioning the free end of an S-shaped spring clip 330 or 430 in contact with the most-distal tooth gap G of the rack facet 141, or any gap of the rack. Methods may include loading the spring clip 330 or 430 under force when it is resting in a gap. Methods may include, after the syringe 10 is loaded, in a metered state, and readied for injections, incrementing injections for the loaded neurotoxin to a patient. Methods may include creating audible and/or tactile feedback for each incremented injection. Methods may include incrementing injections by pushing the plunger 100 forward (distal direction) to increment medicament injections and advancing the free end of a spring clip 330 or 430 of the syringe 10 to the another (e.g., next) tooth 145 of the rack to produce an audible snap for each increment and/or tactile feedback for each increment. Methods may include dispensing neurotoxin by injecting neurotoxin in increments of 0.02 mL such that all or substantially all of the amount of neurotoxin solution is dispensed from the syringe 10 when the plunger 100 of the syringe 10 is at its most-distal position. Methods may include dispensing less than about 0.02 mL of the amount of neurotoxin solution from the syringe 10 when the plunger 100 of the syringe 10 is at its most-distal position. Methods may include dispensing all but about 0.009 mL to about 0.02 mL, including intervening values, e.g., 0.015 mL from the syringe 10 when the plunger 100 of the syringe 10 is at its most-distal position. Methods may include connecting, via a luer-lock interface 290, a needle 810 to the syringe 10 to form a syringe-needle assembly, and dispensing substantially all of the amount of neurotoxin solution to a patient through the needle 810 of the syringe-needle assembly. Method may include using a syringe-needle assembly that has a dead volume of less than about 0.02 mL. Methods may include dispensing all but about 0.01 mL to about 0.025 mL of neurotoxin solution to a patient through the needle 810 of the syringe-needle assembly, e.g., all but about 0.016 mL. Methods may include dispensing in increments of 0.02 mL per increment or 0.025 mL per increment. Methods may include using a 1 mL volume syringe 10, loading it with 1 mL of neurotoxin solution and injecting neurotoxin in increments of 0.02 mL or 0.025 mL such that all or substantially all, e.g., less than about 0.02 mL, e.g., all but about 0.009 mL to about 0.02 mL, including intervening values, e.g., all but about 0.015 mL or 0.016 mL, is dispensed from the syringe 10 when the plunger 100 of the syringe 10 is at its most-distal position.

### EXAMPLE 1

Use of a botulinum toxin injection kit 700 is described. The kit 700 contained four-1 mL syringes 10, each syringe 10 set to a free (withdraw) operational state and the free end of each S-shaped spring clip resting in a relief; four-33G×⅜ inch hypodermic needle elements 800 with needle shields 832, and one-20 mSm medicament vial adapter 900. The kit 700 was used for a single use to inject botulinum neurotoxin into a single patient. In this example, a 100 unit (U) vial of botulinum neurotoxin was used. Each syringe 10 had a bias member in the form of a polycarbonate S-shaped spring clip 430. The vial adapter 900 was wiped with sterile alcohol before being connected to the botulinum toxin vial. The adapter 900 was placed over the botulinum toxin vial and snapped onto the vial to pierce the vial septum.

The botulinum toxin was reconstituted by addition of diluent to the vial by connecting a sterile luer-lock needle-less syringe 10 containing diluent to the adapter. The needle-less syringe 10 was held by the barrel 200 and the syringe luer 290 was pushed into the adapter valve and twisted clockwise. Diluent was injected into the vial using the needleless syringe 10, and the syringe 10 was disconnected from the vial adapter by twisting it counterclockwise. In this example, 2 mL of diluent was added to the 100 Unit (U) vial of botulinum toxin to reconstitute the botulinum toxin. For the given concentration, 0.02 mL=1 Unit.

A sterile, unused syringe 10 was then removed from the kit and the free operational state was visually confirmed by observing the relative position of the asymmetrical plunger flange 132 relative to the grip 300. Once confirmed, the syringe 10 was connected to the adapter valve 900 without a needle by holding the syringe barrel 200 and pushing the syringe luer 290 into the valve and twisting it clockwise. The botulinum toxin vial was inverted so that the vial was above the syringe 10, and botulinum was drawn into the syringe 10 by pulling back on the plunger 100. Pulling back on the plunger portion 100 also moved the free end of the spring clip 330 or 430 away from the relief 180 and loaded it under force. Air was pushed back into the vial to ensure no air bubbles were in the syringe 10. The syringe 10 was held by one hand and the vial adapter 900 in the other, and the syringe 10 was twisted counterclockwise to disconnect the syringe 10 form the vial adapter 900.

A needle element 800 enclosed in a needle protector 830 was removed from the kit 700 and the needle hub cover 834 was separated from the protector 830 to expose the needle hub. The syringe 10 was held and the needle hub was inserted into the luer-lock tip 290 of the syringe 10 and tightened by turning it clockwise until it was fully engaged.

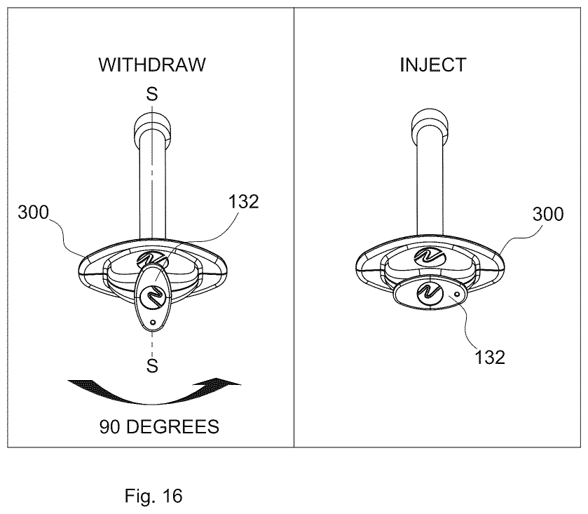

The plunger 100 of the syringe 10 was rotated 90 degrees to change the syringe 10 operating state from the free operating state (withdraw) to the metered operating state (inject), as shown in FIG. 16. Accordingly, the syringe 10 was held in one hand and the plunger flange 132 in the other, and the plunger flange 132 was twisted 90 degrees to align the long dimension of the plunger flange 132 with the long dimension of the syringe grip 300.

The needle shield 832 was removed from the needle assembly 800 by holding the syringe 10 body in one hand and the shield 832 in the other and removing the cap by pulling apart the shield 832 from the needle assembly 800, i.e., without twisting.

Botulinum toxin was incrementally dispensed from the syringe 10 by pushing the plunger 100 forward (distally). The spring clip 330 or 430 provided tactile and audible snap feedback for each increment to control botulinum toxin delivery. In this example, each snap (increment) indicated when 0.02 mL of the botulinum solution was injected, and five snaps (increments) indicated when 0.1 mL had been injected. Because 0.02 mL=1 Unit in this example, 1 snap=1 Unit and 5 snaps =5 Units.

Example 1 Volume/Dose Equivalence

| Volume (mL) | Dose (Units) |
|---|---|
| 0 | 0 |
| 0.1 | 5 |
| 0.2 | 10 |
| 0.3 | 15 |
| 0.4 | 20 |
| 0.5 | 25 |
| 0.6 | 30 |
| 0.7 | 35 |
| 0.8 | 40 |
| 0.9 | 45 |
| 1.0 | 50 |

Additional syringes of the kit 700 were prepared and used in the manner described above. Accordingly, the entire contents of the reconstituted 100 Unit vial of botulinum toxin was dispensed using the kit contents.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. These embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the scope of the claims by features, functions, steps, or elements that are not within that scope.

1-70. (canceled)

71. An injection system comprising a syringe, the syringe comprising:
a barrel portion and a plunger portion at least partially received within the barrel portion;
the barrel portion comprising a proximal barrel flange, a medial cylinder having a consistent diameter, and a distal reduced-diameter neck;
the plunger portion comprising a proximal plunger flange, a medial shaft, and a distal piston;
the piston comprising a resilient material and configured to provide a seal with an interior surface of the medial cylinder;
the barrel flange comprising an at least partially hollow housing with a bias member retained within the housing and with an end of the bias member free to translate in position within the housing; and,
the medial shaft comprising a four-faceted configuration with adjacent facets disposed at right angles to one another,
wherein the syringe is configured to be in a metered state or a free state, wherein at least two of the facets are rack facets, wherein each rack facet comprises teeth configured to consistently and progressively engage with the end of the bias member during axial translation in a periodic fashion when in syringe is in the metered state; and,
wherein at least one of the facets is a free facet without teeth and is configured to consistently and without interruption contact the end of the bias member during axial translation when in the syringe is in the free state.

72. The injection system of claim 71, wherein the medial shaft further comprises a relief to avoid engagement of the end of the bias member with non-relief portions of the medial shaft when the plunger portion is fully advanced within the barrel portion.

73. The injection system of claim 72, wherein the relief is provided on a free facet.

74. The injection system of claim 73, wherein the relief is configured to maintain the bias member in an unbiased or low biased state when the free end of the bias member is engaged with the relief.

75. The injection system of claim 74, wherein the bias member is a spring clip.

76. The injection system of claim 75, wherein the spring clip is an S-shaped spring clip.

77. The injection system of claim 76, wherein the S-shaped spring clip is composed of plastic.

78. The injection system of claim 77, wherein two facets of the plunger portion define two free states.

79. The injection system of claim 77, wherein only one facet of the plunger portion defines a free state and three facets define the metered state.

80. The injection system of claim 79, wherein the plunger portion includes a distal-most tip configuration to closely fit within an interior surface the neck.

81. The injection system of claim 80, wherein an exterior of the neck is configured with a luer-lock interface.

82. The injection system of claim 81, further comprising a vial adapter and a needle element, wherein the vial adapter and the needle element each comprise a luer-lock interface complementary to the luer-lock interface of the neck.

83. The injection system of claim 82, wherein the syringe is connected to the vial adapter and forms a syringe-vial assembly, and wherein the syringe-vial adapter assembly has a near-zero dead volume.

84. The injection system of claim 82, wherein the syringe is connected to the needle and forms a syringe-needle assembly, and wherein the syringe-needle assembly has a near-zero dead volume.

85. The injection system of claim 84, wherein the syringe-needle assembly has a near-zero dead volume of about 0.01 mL to about 0.025 mL

86. The injection system of claim 85, wherein the syringe-needle assembly has a near-zero dead volume of about 0.016 mL.

87. The injection system of claim 84, wherein the syringe-needle assembly has a near-zero dead volume of less than about 0.02 mL.

88. The injection system of claim 87, wherein the syringe has a near-zero dead volume of about 0.009 mL to about 0.02 mL.

89. The injection system of claim 88, wherein the syringe has a near-zero dead volume of about 0.015 mL.

90. The injection system of any of claims 89, wherein the teeth are uniformly spaced apart.

91. The injection system of claim 90, wherein the teeth are spaced apart about 0.045 inches from each other.

92. The injection system of claim 91, wherein each tooth has a depth of about 0.02 inches.

93. The injection system of claim 92, wherein each tooth comprises a proximal-facing surface and a distal-facing surface, wherein the distal-facing surfaces comprises a 90-degree angle.

94. The injection system of claim 93, wherein the proximal-facing surfaces comprises a tooth angle of about 135 degrees.

95. The injection system of claim 94, wherein the medial shaft comprises a bias member relief.

96. The injection system of claim 95, wherein the depth of the bias member relief is greater than the depth of the teeth.

97. The injection system of claim 94, further comprising an anti-rotation mechanism.

98. The injection system of claim 97, wherein the anti-rotation mechanism is configured to prevent rotation of the plunger portion relative to the barrel portion by applying resistance to the plunger portion, wherein the anti-rotation mechanism is further configured to permit the rotation of the plunger portion relative to the barrel portion by application of at least a threshold amount of torque to the plunger portion, and wherein the anti-rotation mechanism does not require any additional steps by a user to activate a clip or a pin after rotation to prevent rotation.

99. The injection system of claim 98, wherein the at least a threshold amount of torque is between about 0.1 lbf-in to about 1.0 lbf-in.

100. The injection system of any of claims 99, wherein the anti-rotation mechanism comprises one or more pairs of cooperating gripping ribs and a channel between the gripping ribs, and wherein the one or more pairs of gripping ribs are configured to apply resistance to the plunger portion to prevent the plunger portion from rotational movement.

101. The injection system of claim 100, wherein the anti-rotation mechanism further comprises two pairs of cooperating gripping ribs.

102. The injection system of claim 101, wherein the anti-rotation mechanism is integrated with the bias member to form an anti-rotation-bias assembly.

103. The injection system of claim 102, wherein the anti-rotation mechanism further comprises a first leg portion and a second leg portion, wherein the first leg portion forms a first pair of the two pairs of cooperating gripping ribs and the second leg portion forms a second pair of the two pairs of cooperating gripping ribs.

104. The injection system of claim 103, wherein the first leg portion and the second leg portion are connected together by a bridge portion, wherein the first leg portion and the second leg portion extend radially from a top surface of the bias member to form the channel.

105. The injection system of claim 101, wherein the gripping ribs are configured to permit rotational movement of the plunger portion when at least a threshold amount of torque of between about 0.1 lbf-in to about 1.0 lbf-in is applied to the plunger portion to overcome the anti-rotational resistance.

106. The injection system of any of claims 105, wherein the bias member is configured to strain in two primary areas when under load.
